(19) United States
(12) Patent Application Publication
(10) Pub. No.: US 2026/0242791 A1
(43) Pub. Date: Aug. 20, 2026

(54) MIRNA-484 INHIBITORS AND USES THEREOF

(71) Applicants: BIORCHESTRA CO., LTD., Daejeon (KR); SK BIOPHARMACEUTICALS CO., LTD., Seongnam (KR)

(72) Inventors: Jin-Hyeob RYU, Daejeon (KR); Ha Na JANG, Daejeon (KR); Jongyeob LIM, Daejeon (KR); Yeon-Joo JUNG, Daejeon (KR); Young Jin PARK, Daejeon (KR); Insun KIM, Daejeon (KR); Yu Na LIM, Daejeon (KR); Howon KANG, Seongnam (KR); Chanmi JOUNG, Seongnam (KR); Eunju CHOI, Seongnam (KR); Ah Hyung PARK, Seongnam (KR); Chaehyun YOOK, Seongnam (KR); Seokhwan CHO, Seongnam (KR); Yujin SHIN, Seongnam (KR); So-Young GIL, Seongnam (KR)

(73) Assignees: BIORCHESTRA CO., LTD., Daejeon (KR); SK BIOPHARMACEUTICALS CO., LTD., Seongnam, GG (KR)

(21) Appl. No.: 19/469,576

(22) PCT Filed: Mar. 29, 2024
(86) PCT No.: PCT/IB2024/053115

§ 371 Date: Sep. 26, 2025

Related U.S. Application Data
(60) Provisional application No. 63/493,526, filed on Mar. 31, 2023.

(51) Int. Cl. C12N 15/113; A61K 31/7105; A61K 31/7115; A61K 31/712; A61K 31/7125; A61K 45/06; A61P 25/08

(52) U.S. Cl. CPC C12N 15/113; A61K 31/7105; A61K 31/7115; A61K 31/712; A61K 31/7125; A61K 45/06; A61P 25/08; C12N 2310/113; C12N 2310/141; C12N 2310/315; C12N 2310/321; C12N 2310/3341; C12N 2310/335

(57) ABSTRACT

The present disclosure includes the use of a miR-484 inhibitor for treating a disease or disorder associated with the increased level of miR-484. In some aspects, the miR-484 inhibitor is capable of reducing the expression of a gene and/or protein associated with miR-484.

### CROSS REFERENCE TO EARLIER FILED APPLICATIONS

This Application claims the priority benefit of U.S. Provisional Application No. 63/493,526, filed on Mar. 31, 2023. The entire disclosure of the above-referenced application is incorporated herein by reference in its entirety.

### REFERENCE TO ELECTRONICALLY FILED SEQUENCE LISTING

The content of the electronically submitted sequence listing (Name: 4366_069PC01_SequenceListing_ST26.xml; Size: 174,839 bytes; and Date of Creation: Mar. 20, 2024) is herein incorporated by reference in its entirety.

### FIELD OF THE DISCLOSURE

The present disclosure provides a miR-484 inhibitor (e.g., polynucleotide encoding a nucleotide molecule comprising at least one miR-484 binding site) for the treatment of diseases or disorders.

### BACKGROUND OF THE DISCLOSURE

Epilepsy, or seizure disorder, is a chronic neurological condition characterized by recurrent seizures that begin in the brain and are associated with excessive or abnormal synchronization of neural activity.

The reasons why susceptible individuals develop epilepsy are not completely understood and there are many possible causes. Some epilepsy sufferers require treatment for many years to keep seizures under control. Treatment of epilepsy typically includes medication, and sometimes neurosurgical intervention (lesioning). Electrical stimulation of the vagus nerve using an implantable stimulator has proven successful in reducing the severity and/or frequency of seizures in some cases. There have also been reports of attempts to treat epilepsy by direct electrical stimulation of the brain (DBS). In DBS (deep brain stimulation), neurons in the immediate vicinity of the electrodes are induced to fire (i.e., are recruited) by the electrical stimulus to modify the electrical activity in that area of the brain.

However, alternative ways to treat patients suffering from an epilepsy that is not sufficiently responsive to conventional therapies are needed.

### BRIEF SUMMARY OF THE DISCLOSURE

The present disclosure is directed to an oligonucleotide comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one nucleic acid analog. In some aspects, the at least one nucleic acid analog is a modified nucleic acid at the sugar position.

In some aspects, the at least one nucleic acid analog is selected from Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleic acid (cEt), 2′-O-methyl nucleic acid (2′-OMe), 2′-O-methoxyethyl nucleic acid (2′-MOE), or any combination thereof. In some aspects, the at least one nucleic acid analog is a nucleic acid comprising a bicyclic sugar. In some aspects, the at least one nucleic acid analog comprising a bicyclic sugar is selected from cEt, 2′,4′-constrained 2′-O-methoxyethyl nucleic acid (cMOE), LNA, α-L-LNA, β-D-LNA, 2′-0,4′-C-ethylene-bridged nucleic acids (ENA), amino-LNA, oxy-LNA, or thio-LNA. In some aspects, the at least one nucleic acid analog is 2′-MOE.

In some aspects, the contiguous nucleotide sequence comprises 5′-GAGCCUG-3′. In some aspects, the oligonucleotide further comprises at least 1 nucleic acid, at least 2 nucleic acids, at least 3 nucleic acids, at least 4 nucleic acids, at least 5 nucleic acid, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 5′ of the contiguous nucleotide sequence. In some aspects, the oligonucleotide further comprises at least 1 nucleic acid, at least 2 nucleic acids, at least 3 nucleic acids, at least 4 nucleic acids, at least 5 nucleic acids, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 3′ of the contiguous nucleotide sequence.

In some aspects, the oligonucleotide comprises the sequence selected from the group consisting of: 5′-GAGCCUG-3′, 5′-GAGCCUGA-3′, 5′-UGAGCCUG-3′, 5′-CUGAGCCUG-3′, 5′-ACUGAGCCUG-3′ (SEQ ID NO: 6), 5′-GACUGAGCCUG-3′ (SEQ ID NO: 7), 5′-GGACUGAGCCUG-3′ (SEQ ID NO: 8), 5′-GGGACUGAGCCUG-3′ (SEQ ID NO: 9), 5′-GGGGACUGAGCCUG-3′ (SEQ ID NO: 10), 5′-AGGGGACUGAGCCUG-3′ (SEQ ID NO: 11), 5′-GAGGGGACUGAGCCUG-3′ (SEQ ID NO: 12), 5′-GGAGGGGACUGAGCCUG-3′ (SEQ ID NO: 13), 5′-GGGAGGGGACUGAGCCUG-3′ (SEQ ID NO: 14), 5′-CGGGAGGGGACUGAGCCUG-3′ (SEQ ID NO: 15); 5′-UCGGGAGGGGACUGAGCCUG-3′ (SEQ ID NO: 16), 5′-AUCGGGAGGGGACUGAGCCUG-3′ (SEQ ID NO: 17), 5′-UGAGCCUGA-3′, 5′-CUGAGCCUGA-3′ (SEQ ID NO: 19), 5′-ACUGAGCCUGA-3′ (SEQ ID NO: 20), 5′-GACUGAGCCUGA-3′ (SEQ ID NO: 21), 5′-GGACUGAGCCUGA-3′ (SEQ ID NO: 22), 5′-GGGACUGAGCCUGA-3′ (SEQ ID NO: 23), 5′-GGGGACUGAGCCUGA-3′ (SEQ ID NO: 24), 5′-AGGGGACUGAGCCUGA-3′ (SEQ ID NO: 25), 5′-GAGGGGACUGAGCCUGA-3′ (SEQ ID NO: 26), 5′-GGAGGGGACUGAGCCUGA-3′ (SEQ ID NO: 27), 5′-GGGAGGGGACUGAGCCUGA-3′ (SEQ ID NO: 28), 5′-CGGGAGGGGACUGAGCCUGA-3′ (SEQ ID NO: 29), 5′-UCGGGAGGGGACUGAGCCUGA-3′ (SEQ ID NO: 30), and 5′-AUCGGGAGGGGACUGAGCCUGA-3′ (SEQ ID NO: 31).

In some aspects, the oligonucleotide comprises the sequence selected from the group consisting of: 5′-GAGCCTG-3′, 5′-GAGCCTGA-3′, 5′-TGAGCCTG-3′, 5′-CTGAGCCTG-3′, 5′-ACTGAGCCTG-3′ (SEQ ID NO: 36), 5′-GACTGAGCCTG-3′ (SEQ ID NO: 37), 5′-GGACTGAGCCTG-3′ (SEQ ID NO: 38), 5′-GGGACTGAGCCTG-3′ (SEQ ID NO: 39), 5′-GGGGACTGAGCCTG-3′ (SEQ ID NO: 40), 5′-AGGGGACTGAGCCTG-3′ (SEQ ID NO: 41), 5′-GAGGGGACTGAGCCTG-3′ (SEQ ID NO: 42), 5′-GGAGGGGACTGAGCCTG-3′ (SEQ ID NO: 43), 5′-GGGAGGGGACTGAGCCTG-3′ (SEQ ID NO: 44), 5′-CGGGAGGGGACTGAGCCTG-3′ (SEQ ID NO: 45); 5′-TCGGGAGGGGACTGAGCCTG-3′ (SEQ ID NO: 46), 5′-ATCGGGAGGGGACTGAGCCTG-3′ (SEQ ID NO: 47), 5′-TGAGCCTGA-3′, 5′-CTGAGCCTGA-3′ (SEQ ID NO: 49), 5′-ACTGAGCCTGA-3′ (SEQ ID NO: 50), 5′-GACTGAGCCTGA-3′ (SEQ ID NO: 51), 5′-GGACTGAGCCTGA-3′ (SEQ ID NO: 52), 5′-GGGACTGAGCCTGA-3′ (SEQ ID NO: 53), 5′-GGGGACTGAGCCTGA-3′ (SEQ ID NO: 54), 5′-AGGGGACTGAGCCTGA-3′ (SEQ ID NO: 55), 5′-GAGGGGACTGAGCCTGA-3′ (SEQ ID NO: 56), 5′-GGAGGGGACTGAGCCTGA-3′ (SEQ ID NO: 57), 5′-GGGAGGGGACTGAGCCTGA-3′ (SEQ ID NO: 58), 5′-CGGGAGGGGACTGAGCCTGA-3′ (SEQ ID NO: 59), 5′-TCGGGAGGGGACTGAGCCTGA-3′ (SEQ ID NO: 60), and 5′-ATCGGGAGGGGACTGAGCCTGA-3′ (SEQ ID NO: 61).

In some aspects, the oligonucleotide has at least about 50%, at least about 55%, at least about 60%, at least about 65%, at least about 70%, at least about 75%, at least about 80%, at least about 85%, at least about 90%, at least about 95% sequence identity or at least about 98% sequence identity to 5′-AUCGGGAGGGGACUGAGCCUGA-3′ (SEQ ID NO: 31) or 5′-ATCGGGAGGGGACTGAGCCTGA-3′ (SEQ ID NO: 61).

In some aspects, the oligonucleotide has at least about 90% sequence similarity to 5′-AUCGGGAGGGGACUGAGCCUGA-3′ (SEQ ID NO: 31) or 5′-ATCGGGAGGGGACTGAGCCTGA-3′ (SEQ ID NO: 61).

In some aspects, the oligonucleotide comprises the nucleotide sequence as set forth in 5′-AUCGGGAGGGGACUGAGCCUGA-3′ (SEQ ID NO: 31) or 5′-ATCGGGAGGGGACTGAGCCTGA-3′ (SEQ ID NO: 61) with one substitution or two substitutions.

In some aspects, the oligonucleotide has the nucleotide sequence as set forth in 5′-AUCGGGAGGGGACUGAGCCUGA-3′ (SEQ ID NO: 31) or 5′-ATCGGGAGGGGACTGAGCCTGA-3′ (SEQ ID NO: 61).

In some aspects, the oligonucleotide comprises the nucleotide sequence as set forth in 5′-AUCGGGAGGGGACUGAGCCUGA-3′ (SEQ ID NO: 62), wherein one or more of the bolded letters is a nucleic acid analog.

In some aspects, the nucleotide sequence comprises 5′-AUCGGGAGGGGACUGAGCCUGA-3′ (SEQ ID NO: 63), and wherein one or more of the bolded letters are 2′-O-methoxyethyl nucleic acids.

In some aspects, the disclosure comprises an oligonucleotide comprising the contiguous nucleotide sequence as set forth in 5′-AUCGGGAGGGGACUGAGCCUGA-3′ (SEQ ID NO: 64), wherein the bolded letters are 2′-O-methoxyethyl nucleic acid.

In some aspects, the contiguous nucleotide sequence comprises one or more 5′ methyl pyrimidines. In some aspects, the oligonucleotide comprises the contiguous nucleotide sequence as set forth in 5′-AUCGGGAGGGGACUGAGCCUGA-3′ (SEQ ID NO: 65), wherein one or more of the double underlined letters are 5-methyl pyrimidines.

In some aspects, the oligonucleotide comprises the contiguous nucleotide sequence as set forth in 5′-AUCGGGAGGGGACUGAGCCUGA-3′ (SEQ ID NO: 66), wherein the double underlined letters are 5-methyl pyrimidines.

In some aspects, the disclosure comprises an oligonucleotide comprising the contiguous nucleotide sequence as set forth in 5′-AUCGGGAGGGGACUGAGCCUGA-3′ (SEQ ID NO: 67), wherein the bolded letters 2′-O-methoxyethyl nucleic acids and the double underlined letters are 5-methyl pyrimidines.

In some aspects, the oligonucleotide further comprises a backbone modification. In some aspects, the backbone modification is a phosphorodiamidate morpholino oligomer (PMO) and/or phosphorothioate (PS) modification. In some aspects, each of the nucleotides are modified with PS modifications.

In some aspects, the oligonucleotide is capable of reducing an mRNA level in a cell, e.g., a SH-SY5Y or BV-2 cell, at least about one fold, at least two folds, at least three folds, at least four folds, at least five folds, at least six folds, at least seven folds, at least eight folds, at least nine folds, or at least ten folds compared to a cell that is not treated with the oligonucleotide, wherein the mRNA encodes a proline-rich transmembrane protein 2 (PRRT2), an inward rectifier potassium channel 2 (KCNJ2), an activated CDC42 kinase 1 (TNK2), an ataxin-1-like (ATXN1), a V-type proton ATPase 116 kDa subunit a 2 (ATP6VOA2), a disintegrin and metalloproteinase domain-containing protein 10 (ADAM10), a protocadherin-19 (PCDH19), or any combination thereof.

In some aspects, the oligonucleotide is capable of reducing a protein expression in a cell, e.g., a SH-SY5Y cell, at least about one fold, at least two folds, at least three folds, at least four folds, at least five folds, at least six folds, at least seven folds, at least eight folds, at least nine folds, or at least ten folds compared to a cell that is not treated with the oligonucleotide, wherein the protein comprises a proline-rich transmembrane protein 2 (PRRT2), an inward rectifier potassium channel 2 (KCNJ2), an activated CDC42 kinase 1 (TNK2), an ataxin-1-like (ATXN1), a V-type proton ATPase 116 kDa subunit a 2 (ATP6VOA2), a disintegrin and metalloproteinase domain-containing protein 10 (ADAM10), a protocadherin-19 (PCDH19), or any combination thereof.

In some aspects, the oligonucleotide is incorporated in a delivery agent for delivery. In some aspects, the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the oligonucleotide of the disclosure comprises an antisense oligonucleotide.

In some aspects, the disclosure comprises a delivery agent comprising the oligonucleotide disclosed herein. In some aspects, the delivery agent is a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the disclosure comprises a pharmaceutical composition comprising (i) the oligonucleotide disclosed herein or the delivery agent comprising the oligonucleotide disclosed herein and (ii) a pharmaceutically acceptable carrier.

In some aspects, the disclosure comprises a method of preparing the oligonucleotide disclosed herein, the delivery agent disclosed herein, and the pharmaceutical composition disclosed herein.

In some aspects, the disclosure comprises a method of reducing an mRNA level in a cell, e.g., a SH-SY5Y cell, comprising contacting the cell with the oligonucleotide disclosed herein, wherein the mRNA encodes a proline-rich transmembrane protein 2 (PRRT2), an inward rectifier potassium channel 2 (KCNJ2), an activated CDC42 kinase 1 (TNK2), an ataxin-1-like (ATXN1), or any combination thereof. In some aspects, the mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

In some aspects, the disclosure comprises a method of reducing a protein expression in a cell, e.g., a SH-SY5Y cell, comprising contacting the cell with the oligonucleotide disclosed herein, wherein the protein comprises a V-type proton ATPase 116 kDa subunit a 2 (ATP6V0A2), a disintegrin and metalloproteinase domain-containing protein 10 (ADAM10), a protocadherin-19 (PCDH19), or any combination thereof. In some aspects, the protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

In some aspects, the disclosure comprises a method of reducing an occurrence, a duration, or symptom of a seizure or an epilepsy in a subject in need thereof comprising administering an oligonucleotide comprising a contiguous nucleotide sequence of 10 to 30 nucleotides in length that are complementary to a nucleic acid sequence within miRNA-484 (5′-UCAGGCUCAGUCCCCUCCCGAU-3′ (SEQ ID NO: 1)).

In some aspects, the disclosure comprises a method of treating an epilepsy or a seizure in a subject in need thereof comprising administering an oligonucleotide comprising a contiguous nucleotide sequence of 10 to 30 nucleotides in length that are complementary to a nucleic acid sequence within miRNA-484 (5′-UCAGGCUCAGUCCCCUCCCGAU-3′ (SEQ ID NO: 1)). In some aspects, the epilepsy comprises 1) focal onset seizure: atonic seizures, automatisms, clonic seizures, epileptic spasms, hyperkinetic seizures, myoclonic seizures, tonic seizures, autonomic seizures, behavior arrest, cognitive seizures, emotional seizures, sensory seizures. Focal onset seizure may be divided into focal onset aware seizure (simple partial seizure) and focal onset impaired awareness seizure (complex partial seizure) according to absence and presence of consciousness during seizure activities; 2) generalized onset seizure: myoclonic seizures, myoclonic-tonic-clonic seizures, tonic-clonic seizures, tonic seizure, clonic seizure, myoclonic-atonic seizure, typical absence seizures, atypical absence seizures, eyelid myoclonia; 3) unknown onset seizure (tonic-clonic seizure, epileptic spasms, behavior arrest), or 4) unclassified seizures.

In some aspects, the oligonucleotide in the method is delivered in a delivery agent. In some aspects, the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the method comprises administering the delivery agent comprising the oligonucleotide disclosed herein or the pharmaceutical composition comprising the oligonucleotide disclosed herein.

In some aspects, the oligonucleotide is administered intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracisternally, intracapsularly, intratumorally, topically, or any combination thereof.

### BRIEF DESCRIPTION OF THE DRAWINGS/FIGURES

FIG. 1 shows unmodified (naked) miR-484 antisense oligonucleotide (ASO) (top ASO) and modified miR-484 ASO (bottom ASO). The modified ASO is modified in which the sugars of the first, second, third, 13^{th}, 14^{th}, 18^{th}, 19^{th}, 20^{th}, 21^{st}, and 22^{nd} nucleosides are modified with 2′-O-methoxyethyl, and the bases of 2^{nd}, 3^{rd}, 13^{th}, 14^{th}, 18^{th}, 19^{th}, and20^{th} nucleotides are modified with 5-methyl pyrimidine. Each nucleotide backbone bond is phosphorothioate bond. FIG. 1 also shows unmodified (naked) RNA monomer structure, phosphodiester backbone, phosphorothioate backbone, and a 2′-O-methoxyethyl monomer structure.

FIGS. 2A-2C show the reduction of miR-484 level in SH-SY5Y human cell line by unmodified miR-484 ASO, respectively. FIG. 2A shows the process of unmodified miR-484 ASO transfection in human SH-SY5Y cell line that measures the miR-484 expression levels six hours, 12 hours, and 24 hours after the transfection. FIG. 2B shows a graph of the relative miR-484 expression levels (fold). FIG. 2C shows a table of the miR-484 inhibition in percentage.

FIGS. 2D-2F show the reduction of miR-484 level in BV-2 mouse cell line by unmodified miR-484 ASO, respectively. FIG. 2D shows the process of unmodified miR-484 ASO transfection in BV-2 mouse cell line that measures the miR-484 expression levels six hours, 12 hours, and 24 hours after the transfection. FIG. 2E shows a graph of the relative miR-484 expression levels (fold). FIG. 2F shows a table of the miR-484 inhibition in percentage.

FIGS. 3A-3C show the expression of various candidate genes and proteins in SH-SY5Y 6 hours (FIG. 3A), 12 hours (FIG. 3B), and 24 hours (FIG. 3C) after transfection of biotin-labeled miR-484 mimic. The left bar shows the results by the control, and the right bar shows the results by the miR-484 mimic.

FIGS. 4A and 4B show the expression of various candidate genes and proteins in BV-2 mouse cell line 12 hours (FIG. 4A) and 24 hours (FIG. 4B) after transfection of biotin-labeled miR-484 mimic. The left bar shows the results by the control, and the right bar shows the results by the miR-484 mimic.

FIGS. 5A and 5B show HPD (hippocampal paroxysmal discharge) in kainic acid (KA)-induced mTLE (mesial temporal lobe epilepsy) model mice when unmodified miR-484 ASO was administered. FIG. 5A shows HPD count, and FIG. 5B shows total HPD duration in chronic epilepsy models. Statistical significance was determined using an unpaired t-test. Data is shown as mean±SEM, wherein “***” represents p<0.001, and represents p<0.01 vs Vehicle.

FIG. 6A shows CC₅₀ determined in the 6-Hz test by unmodified miR-484 ASO compared to the vehicle group in pilocarpine-induced SRS model mice. FIG. 6B shows the specific CC₅₀ values, the standard error (SE), and number of data points considered (N) between the vehicle group and the unmodified miR-484 ASO administered group. Statistical significance was determined using a student's t-test. Data is shown as mean SEM, wherein “*” represents p<0.05 vs Vehicle.

FIGS. 7A and 7B show the expression levels of miR-484 in both cortex (FIG. 7A) and hippocampus (FIG. 7B) of pilocarpine-induced SRS model mice at one week post ICV injection in the group of the age-mated naive mice without pilocarpine injection (control), the vehicle group (Pilo-SRS), and the unmodified miR-484 ASO of FIG. 1 administered group (Pilo-SRS+unmodified miR-484 ASO). Statistical significance was determined using a one-way ANOVA with Fisher's LSD. Data is shown as mean±SEM, wherein “****” represents p<0.0001, and “**” represents p<0.01 vs Pilo-SRS.

FIGS. 8A and 8B show HPD (hippocampal paroxysmal discharge) in kainic acid (KA)-induced mTLE model mice when the modified miR-484 ASO of FIG. 1 was administered. FIG. 8A shows HPD count, and FIG. 8B shows total HPD duration in chronic epilepsy models. Data is shown as mean±SEM.

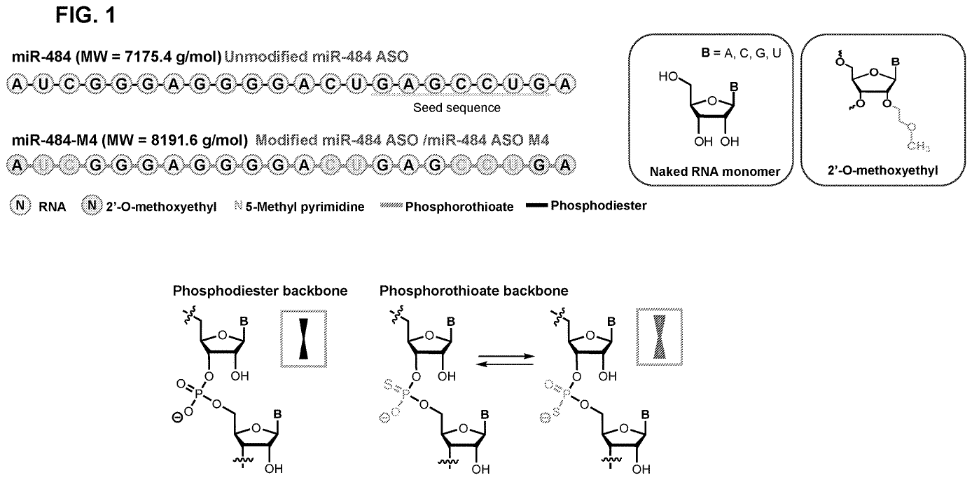

### DETAILED DESCRIPTION OF THE DISCLOSURE

The present disclosure is generally directed to a miR-484 targeting oligonucleotide, e.g., antisense oligonucleotide (ASO), to treat a disease or disorder. In some aspects, a miR-484 targeting oligonucleotide is modified and can have an improved plasma stability and/or in vitro or in vivo cytotoxicity compared to an unmodified targeting oligonucleotide. In some aspects, the miR-484 inhibitors provided herein are useful for treating various diseases or disorders, epilepsy. Additional aspects of the present disclosure are provided throughout the present application.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as a miRNA inhibitor of the present disclosure, into a subject via a pharmaceutically acceptable route. The introduction of a composition, such as a micelle comprising a miRNA inhibitor of the present disclosure, into a subject is by any suitable route, including intracerebrally, intracranially, intracerebroventricularly, intraspinally, intraventricularly, intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “abnormal level” refers to a level (expression and/or activity) that differs (e.g., increased) from a reference subject who does not suffer from a disease or condition described herein). In some aspects, an abnormal level (e.g., inflammasomes) refers to a level that is increased by at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1-fold, at least about 2-fold, at least about 3-fold, at least about 4-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, at least about 750-fold, or at least about 1,000-fold or more compared to the corresponding level in a reference subject (e.g., subject who does not suffer from a disease or condition described herein).

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′->3′),” is complementary to the nucleobase sequence “A-C-T (3′->5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-484 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide. It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

As used herein, the term “homology” refers to the overall relatedness between polymeric molecules, e.g. between nucleic acid molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a miRNA inhibitor of the present disclosure, that has undergone one or more processes of purification. In some aspects, isolating or purifying as used herein is the process of removing, partially removing (e.g., a fraction) of a composition of the present disclosure, e.g., a miRNA inhibitor of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

A “miRNA inhibitor,” as used herein, refers to a compound that can decrease, alter, and/or modulate miRNA expression, function, and/or activity. The miRNA inhibitor can be a polynucleotide sequence (e.g., antisense oligonucleotide, ASO) that is at least partially complementary to the target miRNA sequence, such that the miRNA inhibitor hybridizes to the target miRNA sequence. For instance, in some aspects, a miR-484 inhibitor of the present disclosure comprises a nucleotide sequence encoding a nucleotide molecule that is at least partially complementary to the target miR-484 nucleic acid sequence. In further aspects, the hybridization of the miR-484 inhibitor to the miR-484 sequence decreases, alters, and/or modulates the expression, function, and/or activity of miR-484 (e.g., hybridization results in a decrease in the expression of one or more genes associated with inflammasomes). Unless indicated otherwise, the terms “miRNA inhibitor” or “miR-484 inhibitor,” and can be used interchangeably.

The terms “miRNA,” “miR,” and “microRNA” are used interchangeably and refer to a microRNA molecule found in eukaryotes that is involved in RNA-based gene regulation. The term will be used to refer to the single-stranded RNA molecule processed from a precursor. In some aspects, the term “antisense oligomers” can also be used to describe the microRNA molecules of the present disclosure. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence (e.g., miR-484 inhibitor) that are not matched to a target nucleic acid sequence (e.g., miR-484) according to base pairing rules. While perfect complementarity is often desired, in some aspects, one or more (e.g., 6, 5, 4, 3, 2, or 1 mismatches) can occur with respect to the target nucleic acid sequence. Variations at any location within the oligomer are included. In certain aspects, antisense oligomers of the disclosure (e.g., miR-484 inhibitor) include variations in nucleobase sequence near the termini, variations in the interior, and if present are typically within about 6, 5, 4, 3, 2, or 1 subunit of the 5′ and/or 3′ terminus. In some aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity. In some aspects, a miRNA inhibitor disclosed herein, e.g., a miR-484 inhibitor, can modulate (e.g., decrease, alter, or abolish) miR-484 expression, function, and/or activity, and thereby, can treat epilepsy.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a miRNA inhibitor of the present disclosure, mixed or intermingled with, or suspended in one or more other chemical components, such as pharmaceutically acceptable carriers and excipients. One purpose of a pharmaceutical composition is to facilitate administration of preparations comprising a miRNA inhibitor of the present disclosure to a subject.

The term “polynucleotide,” as used herein, refers to polymers of nucleotides of any length, including ribonucleotides, deoxyribonucleotides, analogs thereof, or mixtures thereof.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure, a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a miR-484 inhibitor disclosed herein (e.g., a polynucleotide encoding a RNA comprising one or more miR-484 binding site) can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. miRNA molecules). Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a miRNA inhibitor of the disclosure (e.g., miR-484 inhibitor), e.g., to decrease abnormal inflammasome activity.

As used herein, the term “therapeutically effective amount” is the amount of reagent or pharmaceutical compound comprising a miRNA inhibitor of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof.

The term “upstream” refers to a nucleotide sequence that is located 5′ to a reference nucleotide sequence.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

II. miRNA-484 Inhibitors Useful for the Present Disclosure

Disclosed herein is an improved antisense oligonucleotide that can inhibit miR-484 activity (miR-484 inhibitor). In some aspects, a miR-484 inhibitor of the present disclosure comprises a nucleotide sequence encoding a nucleotide molecule that comprises at least one miR-484 binding site, wherein the nucleotide molecule does not encode a protein and wherein the miR-484 inhibitor comprises at least one nucleotide analog. In some aspects, a miR-484 inhibitor of the present disclosure comprises a nucleotide sequence encoding a nucleotide molecule that comprises at least one miR-484 binding site, wherein the nucleotide molecule does not encode a protein and wherein the miR-484 inhibitor comprises at least two different nucleic acid analogs. As described herein, in some aspects, the miR-484 binding site in miR-484 inhibitor is at least partially complementary to the target miRNA nucleic acid sequence (i.e., miR-484), such that the miR-484 inhibitor hybridizes to the miR-484 nucleic acid sequence.

In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one nucleic acid analog or at least two different nucleic acid analogs. In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least two different nucleic acid analogs. In some aspects, the different nucleic acid analogs are sugar modified nucleic acid.

In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one nucleic acid analog selected from Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleic acid (cEt), 2′-O-methyl nucleic acid (2′-OMe), 2′-O-methoxyethyl nucleic acid (2′-MOE), and any combination thereof. In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one Locked Nucleic Acid (LNA). In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one 2′-O-methyl nucleic acid (2′-OMe). In some aspects, the present disclosure comprises an oligonucleotide, e.g., an antisense oligonucleotide (ASO), comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), wherein the contiguous nucleotide sequence comprises at least one 2′-O-methoxyethyl nucleic acid (2′-MOE).

In some aspects, the present disclosure is directed to an oligonucleotide, e.g., antisense oligonucleotide (ASO) comprising a contiguous nucleotide sequence of 10 to 30 nucleic acids in length that are complementary to a nucleic acid sequence within miRNA 484 (5′-UCAGGCUCAGUCCCCUCCCGAU-3′ (SEQ ID NO: 1)), wherein the contiguous nucleotide sequence comprises at least one nucleic acid analog that comprises 2′-MOE.

In some aspects, the miR-484 binding site of a miR inhibitor disclosed herein has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence of a miR-484. In some aspects, the miR-484 binding site of a miR inhibitor is fully complementary to the nucleic acid sequence of a miR-484.

The human mature miR-484 has the sequence 5′-UCAGGCUCAGUCCCCUCCCGAU-3′ (SEQ ID NO: 1; miRBase Acc. No. MIMAT0002174). A subsequence of miR-484 5′-GAGCCUG-3′ is the seed sequence.

In some aspects, the miR-484 binding site of a miR inhibitor is a single-stranded polynucleotide sequence that is complementary (e.g., fully complementary) to a sequence of a miR-484 (or a subsequence thereof). In some aspects, the miR-484 subsequence comprises the seed sequence. Accordingly, in certain aspects, the miR-484 binding site of a miR inhibitor has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 49. In certain aspects, the miR-484 binding site of a miR inhibitor is complementary to miR-484 except for 1, 2, 3, 4, 5, 6, 7, 8, 9, or 10 mismatches. In further aspects, the miR-484 binding site of a miR inhibitor is fully complementary to the nucleic acid sequence set forth in SEQ ID NO: 1.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-484 binding site comprises a subsequence that is fully complementary (i.e., 100% complementary) over the entire length of the seed sequence of miR-484.

miRNA sequences and miRNA binding sequences that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name is contemplated also to cover miRNAs or complementary sequences thereof whose sequences are at least about at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-484. In some aspects, miRNA binding sequences of the present disclosure can differ in at least 1, 2, 3, 4, 5, 6, 7, 8, 9, 10 or more nucleotides with respect to those sequence in the sequence listing provided, as long as the modified sequence is still capable of specifically binding to miR-484.

It is also specifically contemplated that any methods and compositions discussed herein with respect to miRNA binding molecules or miRNA can be implemented with respect to synthetic miRNAs binding molecules. It is also understood that the disclosures related to RNA sequences in the present disclosure are equally applicable to corresponding DNA sequences.

In some aspects, a miRNA-484 inhibitor of the present disclosure comprises at least 1 nucleic acid, at least 2 nucleic acids, at least 3 nucleic acids, at least 4 nucleic acids, at least 5 nucleic acids, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 5′ of the nucleotide sequence. In some aspects, a miRNA-484 inhibitor comprises at least 1 nucleic acid, at least 2 nucleic acids, at least 3 nucleic acids, at least 4 nucleic acids, at least 5 nucleic acids, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 3′ of the nucleotide sequence.

In some aspects, a miR-484 inhibitor disclosed herein is about 6 to about 30 nucleic acids in length. In certain aspects, a miR-484 inhibitor disclosed herein is 7 nucleic acids in length. In further aspects, a miR-484 inhibitor disclosed herein is 8 nucleic acids in length. In some aspects, a miR-484 inhibitor is 9 nucleic acids in length. In some aspects, a miR-484 inhibitor of the present disclosure is 10 nucleic acids in length. In certain aspects, a miR-484 inhibitor is 11 nucleic acids in length. In further aspects, a miR-484 inhibitor is 12 nucleic acids in length. In some aspects, a miR-484 inhibitor disclosed herein is 13 nucleic acids in length. In certain aspects, a miR-484 inhibitor disclosed herein is 14 nucleic acids in length. In some aspects, a miR-484 inhibitor disclosed herein is 15 nucleic acids in length. In further aspects, a miR-484 inhibitor is 16 nucleic acids in length. In certain aspects, a miR-484 inhibitor of the present disclosure is 17 nucleic acids in length. In some aspects, a miR-484 inhibitor is 18 nucleic acids in length. In some aspects, a miR-484 inhibitor is 19 nucleic acids in length. In certain aspects, a miR-484 inhibitor is 20 nucleic acids in length. In further aspects, a miR-484 inhibitor of the present disclosure is 21 nucleic acids in length. In some aspects, a miR-484 inhibitor is 22 nucleic acids in length.

In some aspects, a miR-484 inhibitor disclosed herein comprises a nucleotide sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from 5′-GAGCCUG-3′, 5′-GAGCCUGA-3′, 5′-UGAGCCUG-3′, 5′-CUGAGCCUG-3′, 5′-UGAGCCUGA-3′, and SEQ ID NOs: 6-17 and 19-30. In certain aspects, a miR-484 inhibitor comprises a nucleotide sequence selected from the group consisting of 5′-GAGCCUG-3′, 5′-GAGCCUGA-3′, 5′-UGAGCCUG-3′, 5′-CUGAGCCUG-3′, 5′-UGAGCCUGA-3′, and SEQ ID NOs: 6-17 and 19-30, wherein the nucleotide sequence can optionally comprise 1, 2, 3, 4, 5, 6, 7, 8, 9, or 10 mismatches. In some aspects, the miRNA inhibitor of the present disclosure has a plasma stability that exhibits at least about 50%, at least about 60%, at least about 70%, at least about 80%, at least about 90%, at least about 95%, or about 100% recovery after one day, as measured by an HPLC.

In some aspects, the miRNA inhibitor of the present disclosure has a plasma stability that exhibits at least about 60%, at least about 70%, at least about 80%, at least about 90%, at least about 95%, or about 100% recovery after about two days, about three days, about four days, about five days, about six days, about seven days, about eight days, about nine days, or about ten days, as measured by an HPLC.

In some aspects, the miRNA inhibitor of the present disclosure has a plasma stability that exhibits at least about 90%, at least about 95%, or about 100% recovery after about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days, as measured by an HPLC.

In some aspects, a miRNA inhibitor comprises the sequence selected from the group consisting of: 5′-GAGCCUG-3′, 5′-GAGCCUGA-3′, 5′-UGAGCCUG-3′, 5′-CUGAGCCUG-3′, 5′-ACUGAGCCUG-3′ (SEQ ID NO: 6), 5′-GACUGAGCCUG-3′ (SEQ ID NO: 7), 5′-GGACUGAGCCUG-3′ (SEQ ID NO: 8), 5′-GGGACUGAGCCUG-3′ (SEQ ID NO: 9), 5′-GGGGACUGAGCCUG-3′ (SEQ ID NO: 10), 5′-AGGGGACUGAGCCUG-3′ (SEQ ID NO: 11), 5′-GAGGGGACUGAGCCUG-3′ (SEQ ID NO: 12), 5′-GGAGGGGACUGAGCCUG-3′ (SEQ ID NO: 13), 5′-GGGAGGGGACUGAGCCUG-3′ (SEQ ID NO: 14), 5′-CGGGAGGGGACUGAGCCUG-3′ (SEQ ID NO: 15); 5′-UCGGGAGGGGACUGAGCCUG-3′ (SEQ ID NO: 16), 5′-AUCGGGAGGGGACUGAGCCUG-3′ (SEQ ID NO: 17), 5′-UGAGCCUGA-3′, 5′-CUGAGCCUGA-3′ (SEQ ID NO: 19), 5′-ACUGAGCCUGA-3′ (SEQ ID NO: 20), 5′-GACUGAGCCUGA-3′ (SEQ ID NO: 21), 5′-GGACUGAGCCUGA-3′ (SEQ ID NO: 22), 5′-GGGACUGAGCCUGA-3′ (SEQ ID NO: 23), 5′-GGGGACUGAGCCUGA-3′ (SEQ ID NO: 24), 5′-AGGGGACUGAGCCUGA-3′ (SEQ ID NO: 25), 5′-GAGGGGACUGAGCCUGA-3′ (SEQ ID NO: 26), 5′-GGAGGGGACUGAGCCUGA-3′ (SEQ ID NO: 27), 5′-GGGAGGGGACUGAGCCUGA-3′ (SEQ ID NO: 28), 5′-CGGGAGGGGACUGAGCCUGA-3′ (SEQ ID NO: 29), 5′-UCGGGAGGGGACUGAGCCUGA-3′ (SEQ ID NO: 30), and 5′-AUCGGGAGGGGACUGAGCCUGA-3′ (SEQ ID NO: 31), wherein the miRNA inhibitor comprises at least one nucleic acid analog, e.g., 2′-MOE, at least one base modification, e.g., 5-methyl pyrimidine, and/or at least one backbone modification, e.g., PS modification.

In some aspects, a miRNA inhibitor comprises the sequence as set forth in 5′-GGGGACUGAGCCUGA-3′ (SEQ ID NO: 24), wherein the miRNA inhibitor comprises at least one nucleic acid analog, e.g., 2′-MOE. In some aspects, a miRNA inhibitor comprises the sequence as set forth in 5′-AGGGGACUGAGCCUGA-3′ (SEQ ID NO: 25), wherein the miRNA inhibitor comprises at least one nucleic acid analog, e.g., 2′-MOE. In some aspects, a miRNA inhibitor comprises the sequence as set forth in 5′-GAGGGGACUGAGCCUGA-3′ (SEQ ID NO: 26), wherein the miRNA inhibitor comprises at least one nucleic acid analog, e.g., 2′-MOE. In some aspects, a miRNA inhibitor comprises the sequence as set forth in 5′-GGAGGGGACUGAGCCUGA-3′ (SEQ ID NO: 27), wherein the miRNA inhibitor comprises at least one nucleic acid analog, e.g., 2′-MOE. In some aspects, a miRNA inhibitor comprises the sequence as set forth in 5′-GGGAGGGGACUGAGCCUGA-3′ (SEQ ID NO: 28), wherein the miRNA inhibitor comprises at least one nucleic acid analog, e.g., 2′-MOE. In some aspects, a miRNA inhibitor comprises the sequence as set forth in 5′-CGGGAGGGGACUGAGCCUGA-3′ (SEQ ID NO: 29), wherein the miRNA inhibitor comprises at least one nucleic acid analog, e.g., 2′-MOE. In some aspects, a miRNA inhibitor comprises the sequence as set forth in 5′-UCGGGAGGGGACUGAGCCUGA-3′ (SEQ ID NO: 30), wherein the miRNA inhibitor comprises at least one nucleic acid analog, e.g., 2′-MOE. In some aspects, a miRNA inhibitor comprises the sequence as set forth in 5′-AUCGGGAGGGGACUGAGCCUGA-3′ (SEQ ID NO: 31), wherein the miRNA inhibitor comprises at least one nucleic acid analog, e.g., 2′-MOE, at least one base modification, e.g., 5-methyl pyrimidine, and/or at least one backbone modification, e.g., PS modification.

In some aspects, the miRNA inhibitor has the selected from the group consisting of: 5′-GAGCCTG-3′, 5′-GAGCCTGA-3′, 5′-TGAGCCTG-3′, 5′-CTGAGCCTG-3′, 5′-ACTGAGCCTG-3′ (SEQ ID NO: 36), 5′-GACTGAGCCTG-3′ (SEQ ID NO: 37), 5′-GGACTGAGCCTG-3′ (SEQ ID NO: 38), 5′-GGGACTGAGCCTG-3′ (SEQ ID NO: 39), 5′-GGGGACTGAGCCTG-3′ (SEQ ID NO: 40), 5′-AGGGGACTGAGCCTG-3′ (SEQ ID NO: 41), 5′-GAGGGGACTGAGCCTG-3′ (SEQ ID NO: 42), 5′-GGAGGGGACTGAGCCTG-3′ (SEQ ID NO: 43), 5′-GGGAGGGGACTGAGCCTG-3′ (SEQ ID NO: 44), 5′-CGGGAGGGGACTGAGCCTG-3′ (SEQ ID NO: 45); 5′-TCGGGAGGGGACTGAGCCTG-3′ (SEQ ID NO: 46), 5′-ATCGGGAGGGGACTGAGCCTG-3′ (SEQ ID NO: 47), 5′-TGAGCCTGA-3′, 5′-CTGAGCCTGA-3′ (SEQ ID NO: 49), 5′-ACTGAGCCTGA-3′ (SEQ ID NO: 50), 5′-GACTGAGCCTGA-3′ (SEQ ID NO: 51), 5′-GGACTGAGCCTGA-3′ (SEQ ID NO: 52), 5′-GGGACTGAGCCTGA-3′ (SEQ ID NO: 53), 5′-GGGGACTGAGCCTGA-3′ (SEQ ID NO: 54), 5′-AGGGGACTGAGCCTGA-3′ (SEQ ID NO: 55), 5′-GAGGGGACTGAGCCTGA-3′ (SEQ ID NO: 56), 5′-GGAGGGGACTGAGCCTGA-3′ (SEQ ID NO: 57), 5′-GGGAGGGGACTGAGCCTGA-3′ (SEQ ID NO: 58), 5′-CGGGAGGGGACTGAGCCTGA-3′ (SEQ ID NO: 59), 5′-TCGGGAGGGGACTGAGCCTGA-3′ (SEQ ID NO: 60), and 5′-ATCGGGAGGGGACTGAGCCTGA-3′ (SEQ ID NO: 61), wherein the miRNA inhibitor comprises at least one nucleic acid analog, e.g., 2′-MOE. In some aspects, the miRNA inhibitor has the sequence as set forth in 5′-GGAGGGGACTGAGCCTGA-3′ (SEQ ID NO: 57), wherein the miRNA inhibitor comprises at least one nucleic acid analog, e.g., 2′-MOE. In some aspects, the miRNA inhibitor has the sequence as set forth in 5′-GGGAGGGGACTGAGCCTGA-3′ (SEQ ID NO: 58), wherein the miRNA inhibitor comprises at least one nucleic acid analog, e.g., 2′-MOE. In some aspects, the miRNA inhibitor has the sequence as set forth in 5′-CGGGAGGGGACTGAGCCTGA-3′ (SEQ ID NO: 59), wherein the miRNA inhibitor comprises at least one nucleic acid analog, e.g., 2′-MOE. In some aspects, the miRNA inhibitor has the sequence as set forth in 5′-TCGGGAGGGGACTGAGCCTGA-3′ (SEQ ID NO: 60), wherein the miRNA inhibitor comprises at least one nucleic acid analog, e.g., 2′-MOE. In some aspects, the miRNA inhibitor has the sequence as set forth in 5′-ATCGGGAGGGGACTGAGCCTGA-3′ (SEQ ID NO: 61), wherein the miRNA inhibitor comprises at least one nucleic acid analog, e.g., 2′-MOE, at least one base modification, e.g., 5-methyl pyrimidine, and/or at least one backbone modification, e.g., PS modification.

In some aspects, a miRNA inhibitor disclosed herein (i.e., miR-484 inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% sequence identity to 5′-AUCGGGAGGGGACUGAGCCUGA-3′ (SEQ ID NO: 31) or 5′-ATCGGGAGGGGACTGAGCCTGA-3′ (SEQ ID NO: 61), wherein the miRNA inhibitor comprises at least one nucleic acid analog, e.g., 2′ MOE. In some aspects, the miRNA inhibitor comprises a nucleotide sequence that has at least 90% sequence identity to 5′-AUCGGGAGGGGACUGAGCCUGA-3′ (SEQ ID NO: 31) or 5′-ATCGGGAGGGGACTGAGCCTGA-3′ (SEQ ID NO: 61), wherein the miRNA inhibitor comprises at least one nucleic acid analog, e.g., 2′ MOE. In some aspects, the miRNA inhibitor comprises the nucleotide sequence 5′-AUCGGGAGGGGACUGAGCCUGA-3′ (SEQ ID NO: 31) or 5′-ATCGGGAGGGGACTGAGCCTGA-3′ (SEQ ID NO: 61), with one substitution or two substitutions, wherein the miRNA inhibitor comprises at least one nucleic acid analog, e.g., 2′ MOE. In some aspects, the miRNA inhibitor comprises the nucleotide sequence 5′-AUCGGGAGGGGACUGAGCCUGA-3′ (SEQ ID NO: 31) or 5′-ATCGGGAGGGGACTGAGCCTGA-3′ (SEQ ID NO: 61), wherein the miRNA inhibitor comprises at least one nucleic acid analog, e.g., 2′ MOE. In certain aspects, the miRNA inhibitor comprises the nucleotide sequence 5′-AUCGGGAGGGGACUGAGCCUGA-3′ (SEQ ID NO: 31), wherein the miRNA inhibitor comprises at least one nucleic acid analog, e.g., 2′ MOE.

In some aspects, the oligonucleotide comprises the nucleotide sequence as set forth in 5′-AUCGGGAGGGGACUGAGCCUGA-3′ (SEQ ID NO: 62), wherein one or more of the bolded letters is a nucleic acid analog, e.g., 2′-MOE.

In some aspects, the oligonucleotide comprises the nucleotide sequence as set forth in 5′-AUCGGGAGGGGACUGAGCCUGA-3′ (SEQ ID NO: 63), and wherein one or more of the bolded letters are 2′-MOEs.

In some aspects, the oligonucleotide comprises the nucleotide sequence as set forth in 5′-AUCGGGAGGGGACUGAGCCUGA-3′ (SEQ ID NO: 64), and wherein the bolded letters are 2′-MOEs.

In some aspects, the oligonucleotide comprises one or more modified base in the nucleotide sequence. In some aspects, one or more modified bases are 5′ methyl pyrimidines.

In some aspects, the oligonucleotide comprises the contiguous nucleotide sequence as set forth in 5′-AUCGGGAGGGGACUGAGCCUGA-3′ (SEQ ID NO: 65), wherein one or more of the double underlined letters are 5-methyl pyrimidine.

In some aspects, the oligonucleotide comprises the contiguous nucleotide sequence as set forth in 5′-AUCGGGAGGGGACUGAGCCUGA-3′ (SEQ ID NO: 66), wherein the double underlined letters are 5-methyl pyrimidine.

In some aspects, the oligonucleotide comprises the contiguous nucleotide sequence as set forth in 5′-AUCGGGAGGGGACUGAGCCUGA-3′ (SEQ ID NO: 67), wherein the bolded letters are 2′-MOEs and the double underlined letters are 5-methyl pyrimidines.

In some aspects, the oligonucleotide disclosed herein has one or more backbone modification, e.g., phosphothioate bond. In some aspects, the backbone modification is a phosphorodiamidate morpholino oligomer (PMO) and/or phosphorothioate (PS) modification.

In some aspects, the oligonucleotide comprises the nucleotide sequence as set forth in 5′-AUCGGGAGGGGACUGAGCCUGA-3′ (SEQ ID NO: 68), wherein one or more of the bolded letters is a nucleic acid analog, e.g., 2′-MOE, and the backbone of the oligonucleotide is PS modified.

In some aspects, the oligonucleotide comprises the nucleotide sequence as set forth in 5′-AUCGGGAGGGGACUGAGCCUGA-3′ (SEQ ID NO: 69), and wherein one or more of the bolded letters are 2′-MOEs, and the backbone of the oligonucleotide is PS modified.

In some aspects, the oligonucleotide comprises the nucleotide sequence as set forth in 5′-AUCGGGAGGGGACUGAGCCUGA-3′ (SEQ ID NO: 70), and wherein the bolded letters are 2′-MOEs, and the backbone of the oligonucleotide is PS modified.

In some aspects, the oligonucleotide comprises the contiguous nucleotide sequence as set forth in 5′-AUCGGGAGGGGACUGAGCCUGA-3′ (SEQ ID NO: 71), wherein one or more of the double underlined letters are 5-methyl pyrimidine and the backbone of the oligonucleotide is PS modified.

In some aspects, the oligonucleotide comprises the contiguous nucleotide sequence as set forth in 5′-AUCGGGAGGGGACUGAGCCUGA-3′ (SEQ ID NO: 72), wherein the double underlined letters are 5-methyl pyrimidine and the backbone of the oligonucleotide is PS modified.

In some aspects, the oligonucleotide comprises the contiguous nucleotide sequence as set forth in 5′-AUCGGGAGGGGACUGAGCCUGA-3′ (SEQ ID NO: 73), wherein the bolded letters are 2′-MOEs and the double underlined letters are 5-methyl pyrimidines and the backbone of the oligonucleotide is PS modified (ASO M4/miR-484-M4/miR-484 ASO M4).

In some aspects, a miR-484 inhibitor of the present disclosure comprises one miR-484 binding site. In further aspects, a miR-484 inhibitor disclosed herein comprises at least two miR-484 binding sites. In certain aspects, a miR-484 inhibitor comprises three miR-484 binding sites. In some aspects, a miR-484 inhibitor comprises four miR-484 binding sites. In some aspects, a miR-484 inhibitor comprises five miR-484 binding sites. In certain aspects, a miR-484 inhibitor comprises six or more miR-484 binding sites. In some aspects, all the miR-484 binding sites are identical. In some aspects, all the miR-484 binding sites are different. In some aspects, at least one of the miR-484 binding sites is different. In some aspects, all the miR-484 binding sites are miR-484 binding sites. In other aspects, all the miR-484 binding sites are miR-484-5p binding sites. In further aspects, a miR-484 inhibitor comprises at least one miR-484 binding site and at least one miR-484-5p binding site.

In some aspects, the oligonucleotides of the disclosure are capable of reducing an mRNA level in a cell, e.g., a SH-SY5Y cell, wherein the mRNA encodes a proline-rich transmembrane protein 2 (PRRT2), an inward rectifier potassium channel 2 (KCNJ2), an activated CDC42 kinase 1 (TNK2), an ataxin-1-like (A TXN), or any combination thereof. In some aspects, the oligonucleotides of the disclosure are capable of reducing an mRNA level in a cell, e.g., a SH-SY5Y cell, wherein the PRRT2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the KCNJ2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the TNK2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the ATXINI mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

In some aspects, the oligonucleotides of the disclosure are capable of reducing a protein expression in a cell, e.g., a SH-SY5Y cell, wherein the protein comprises a V-type proton ATPase 116 kDa subunit a 2 (ATP6VOA2), a disintegrin and metalloproteinase domain-containing protein 10 (ADAM10), a protocadherin-19 (PCDH19), or any combination thereof.

In some aspects, the oligonucleotides of the disclosure are capable of reducing a ATP6VOA2 protein expression in a cell, e.g., a SH-SY5Y cell, wherein the ATP6VOA2 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing a ADAM10 protein expression in a cell, e.g., a SH-SY5Y cell, wherein the ADAM10 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing a PCDH19 protein expression in a cell, e.g., a SH-SY5Y cell, wherein the PCDH19 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

II.a. Chemically Modified Polynucleotides

In some aspects, a miR-484 inhibitor disclosed herein comprises an oligonucleotide which includes at least one chemically modified nucleoside and/or nucleotide, e.g., 2′ MOE, 5-methyl pyrimidine, and/or phosphorothioate modifications.

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” or “nucleic acid” refers to a nucleoside including a phosphate group. Modified nucleotides can be synthesized by any useful method, such as, for example, chemically, enzymatically, or recombinantly, to include one or more modified or non-natural nucleosides.

Polynucleotides, e.g., oligonucleotides, can comprise a region or regions of linked nucleosides. Such regions can have variable backbone linkages. The linkages can be standard phosphodiester linkages, in which case the oligonucleotides would comprise regions of nucleotides.

The modified oligonucleotides disclosed herein can comprise various distinct modifications. In some aspects, the modified polynucleotides contain two or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, an oligonucleotide of the present disclosure (e.g., a miR-484 inhibitor) comprises at least two different chemical modifications. As used herein, in reference to an oligonucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, an oligonucleotide of the present disclosure (e.g., a miR-484 inhibitor) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure (e.g., a miR-484 inhibitor) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, oligonucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the oligonucleotide (e.g., a miR-484 inhibitor) includes a combination of at least one (e.g., 1, 2, 3, 4, 5, 6, 7, 8, 9, 10, 11, 12, 13, 14, 15, 16, 17, 18, 19, 20 or more) modified nucleobase.

In some aspects, the nucleobases, sugar, backbone linkages, or any combination thereof in an oligonucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### (i) Base Modification

In certain aspects, the chemical modification is at nucleobases in an oligonucleotide of the present disclosure (e.g., a miR-484 inhibitor). In some aspects, the at least one chemically modified nucleic acid comprises a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g., 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the oligonucleotide of the present disclosure (e.g., a miR-484 inhibitor) is uniformly modified (e.g., fully modified, modified throughout the entire sequence) for a particular modification. For example, an oligonucleotide can be uniformly modified with the same type of base modification, e.g., 5-methyl-cytidine (m5C), meaning that all cytosine residues in the polynucleotide sequence are replaced with 5-methyl-cytidine (m5C). Similarly, an oligonucleotide can be uniformly modified for any type of nucleic acid residue present in the sequence by replacement with a modified nucleic acid such as any of those set forth above.

In some aspects, the oligonucleotide of the present disclosure (e.g., a miR-484 inhibitor) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in an oligonucleotide of the present disclosure (e.g., a miR-484 inhibitor) are modified nucleobases.

### (ii) Backbone Modifications

In some aspects, the oligonucleotide of the present disclosure (i.e., miR-484 inhibitor) can include any useful linkage between the nucleosides. Such linkages, including backbone modifications, that are useful in the composition of the present disclosure include, but are not limited to the following: 3-alkylene phosphonates, 3-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

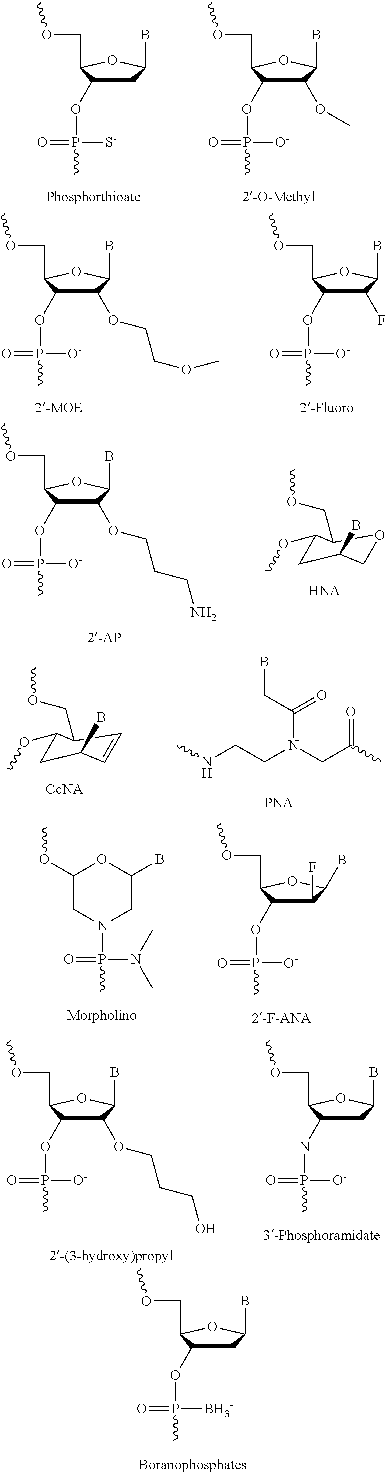

In some aspects, the presence of a backbone linkage disclosed above increase the stability and resistance to degradation of an oligonucleotide of the present disclosure (i.e., miR-484 inhibitor).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in an oligonucleotide of the present disclosure (i.e., miR-484 inhibitor) are modified (e.g., all of them are phosphorothioate).

In some aspects, a backbone modification that can be included in an oligonucleotide of the present disclosure (i.e., miR-484 inhibitor) comprises phosphorodiamidate morpholino oligomer (PMO) and/or phosphorothioate (PS) modification.

(iii) Sugar Modifications

The modified nucleosides and nucleotides which can be incorporated into an oligonucleotide of the present disclosure (i.e., miR-484 inhibitor) can be modified on the sugar of the nucleosides and nucleotides. In some aspects, the sugar modification increases the affinity of the binding of a miR-484 inhibitor to miR-484 nucleic acid sequence. Incorporating affinity-enhancing nucleotide analogues in the miR-484 inhibitor, such as 2′-substituted sugar containing nucleotides, can allow the length and/or the size of the miR-484 inhibitor to be reduced.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in an oligonucleotide of the present disclosure (i.e., miR-484 inhibitor) contain sugar modifications (e.g., 2′ MOE).

In some aspects, 1, 2, 3, 4, 5, 6, 7, 8, 9, 10, 11, 12, 13, 14, 15, 16, 17, 18, 19, 20, 21, or 22 nucleic acid units in an oligonucleotide of the present disclosure are sugar modified (e.g., 2′ MOE).

Generally, RNA includes the sugar group ribose, which is a 5-membered ring having 2′ hydroxyl group. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′-2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, an oligonucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in an oligonucleotide of the present disclosure (i.e., mir-484 inhibitor) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio-LNA or alpha-L-thio-LNA), ENA (such a beta-D-ENA or alpha-L-ENA), or any combination thereof. In further aspects, nucleotide analogues that can be included in an oligonucleotide of the present disclosure (i.e., miR-484 inhibitor) comprises a locked nucleic acid (LNA), an unlocked nucleic acid (UNA), an arabino nucleic acid (ABA), a bridged nucleic acid (BNA), and/or a peptide nucleic acid (PNA).

In some aspects, an oligonucleotide of the present disclosure (i.e., miR-484 inhibitor) can comprise both modified RNA nucleotide analogues (e.g., LNA) and DNA units. In some aspects, a miR-484 inhibitor is a gapmer. See, e.g., U.S. Pat. Nos. 8,404,649; 8,580,756; 8,163,708; 9,034,837; all of which are herein incorporated by reference in their entireties. In some aspects, a miR-484 inhibitor is a micromir. See U.S. Pat. Appl. Publ. No. US20180201928, which is herein incorporated by reference in its entirety.

In some aspects, an oligonucleotide of the present disclosure (i.e., miR-484 inhibitor) can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### III. Vectors and Delivery Systems

In some aspects, the miR-484 inhibitors of the present disclosure can be administered, e.g., to a subject suffering from a disease or condition associated with seizure, using any relevant delivery system known in the art. In some aspects, the delivery system is a vector. Accordingly, in some aspects, the present disclosure provides a vector comprising a miR-484 inhibitor of the present disclosure.

In some aspects, the vector is viral vector. In some aspects, the viral vector is an adenoviral vector or an adenoassociated viral vector. In certain aspects, the viral vector is an AAV that has a serotype of AAV2, AAV3, AAV4, AAV5, AAV6, AAV7, AAV8, AAV9, AAV10, or any combination thereof. In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ~33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY^{T}M and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

Any AAV vector known in the art can be used in the methods disclosed herein. The AAV vector can comprise a known vector or can comprise a variant, fragment, or fusion thereof. In some aspects, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, bovine AAV, shrimp AVV, snake AVV, and any combination thereof.

In some aspects, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain aspects, the AAV vector comprises regions of at least two different AAV vectors known in the art.

In some aspects, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EFla promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancers are present in the AAV alone or together with a promoter disclosed herein. In some aspects, the AAV vector comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A).

In some aspects, a miR-484 inhibitor disclosed herein is administered with a delivery agent comprising the oligonucleotide disclosed herein. Non-limiting examples of delivery agents that can be used include an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a micelle, a viral vector, or a conjugate.

### IV. Pharmaceutical Compositions

In some aspects, the present disclosure also provides pharmaceutical compositions comprising a miR-484 inhibitor disclosed herein (e.g., a polynucleotide or a vector comprising the miR-484 inhibitor) or the delivery agent comprising the oligonucleotide disclosed herein that are suitable for administration to a subject. The pharmaceutical compositions generally comprise a miR-484 inhibitor described herein (e.g., a polynucleotide or a vector) or the delivery agent comprising the oligonucleotide disclosed herein and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a miR-484 inhibitor of the present disclosure or the delivery agent comprising the oligonucleotide disclosed herein. (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

### V. Methods of Treatment

The present disclosure also provides a method of treating a disease or condition that is associated with a miR-484 pathway. In some aspects, the method provides a therapy for a disease or condition that is associated with abnormal (e.g., reduced or increased) proteins and/or genes that are controlled by, regulated by, or associated with miR-484.

In some aspects, a miR-484 inhibitor is capable of treating any epilepsy and epilepsy syndromes.

The term “epilepsy” or “epileptic” in the present disclosure refers to a group of diseases in which epilepsy seizures occur repeatedly and become chronic even though there are no causative factors capable of causing epilepsy. Epilepsy is a neurological disorder that causes recurrent and unprovoked seizures. The epilepsy can be divided into focal onset seizure, generalized onset seizure, and unknown onset seizure. Seizures are defined as a sudden behavioral change due to surges of abnormal and excessive electrical activity in brain. Epilepsy (epileptic disease) caused by diverse etiology is mainly classified based on seizure types, and epilepsy syndromes include not only seizure symptoms but also distinct features such as age, inheritance, EEG pattern, etc (Figure X., Scheffer et al., Epilepsia 58:512-521, 2017).

Seizures are Classified into Three Types:
1) focal onset seizure: atonic seizures, automatisms, clonic seizures, epileptic spasms, hyperkinetic seizures, myoclonic seizures, tonic seizures, autonomic seizures, behavior arrest, cognitive seizures, emotional seizures, sensory seizures. Focal onset seizure may be divided into focal onset aware seizure (simple partial seizure) and focal onset impaired awareness seizure (complex partial seizure) according to absence and presence of consciousness during seizure activities. Moreover, focal onset seizure started in limited area of brain can develop to a secondarily generalized seizure (focal bilateral tonic clonic seizure) by spreading of seizure to both hemispheres of brain;
2) generalized onset seizure: myoclonic seizures, myoclonic-tonic-clonic seizures, tonic-clonic seizures, tonic seizure, clonic seizure, myoclonic-atonic seizure, typical absence seizures, atypical absence seizures, eyelid myoclonia; and
3) unknown onset seizure (tonic-clonic seizure, epileptic spasms, behavior arrest), and 4) unclassified seizures.

Epilepsy syndromes are categorized by its features and seizures as follows:
1) Focal epilepsy syndrome: mesial temporal lobe epilepsy with hippocampal sclerosis, self-limited epilepsy with centrotemporal spikes, self-limited epilepsy with autonomic seizures, self-limited familial neonatal and infantile epilepsy, self-limited (familial) infantile epilepsy, self-limited (familial) neonatal epilepsy, sleep-related hypermotor (hyperkinetic) epilepsy, childhood occipital visual epilepsy, photosensitive occipital lobe epilepsy, familial focal epilepsy with variable foci, familial mesial temporal lobe epilepsy, epilepsy with auditory features;
2) Generalized epilepsy syndrome: juvenile myoclonic epilepsy, childhood absence epilepsy, epilepsy with generalized tonic-clonic seizure alone, juvenile absence epilepsy, epilepsy with eyelid myoclonia, epilepsy with myoclonic absences, myoclonic epilepsy in infancy;
3) Generalized and focal epilepsy syndrome: genetic epilepsy with febrile seizures plus, epilepsy with reading-induced seizures;
4) Syndrome with developmental and epileptic encephalopathy and syndromes with progressive neurological deterioration: infantile epileptic spasm syndrome, developmental and/or epileptic encephalopathy with spike-wave activation in sleep, early infantile developmental and epileptic encephalopathy, Lennox Gastaut syndrome, epilepsy with myoclonic-atonic seizures, Dravet syndrome, epilepsy in infancy with migrating focal seizures, Rasmussen syndrome, febrile infection-related epilepsy syndrome, progressive myoclonic epilepsy, gelastic seizure with hypothalamic hamartoma, hemiconvulsion-hemiplegia epilepsy.

In some aspects, the oligonucleotides of the disclosure are capable of reducing an mRNA level in a cell, e.g., a SH-SY5Y or BV-2 cell, wherein the mRNA encodes a proline-rich transmembrane protein 2 (PRRT2), an inward rectifier potassium channel 2 (KCNJ2), an activated CDC42 kinase 1 (TNK2), an ataxin-1-like (ATXN1), or any combination thereof. In some aspects, the oligonucleotides of the disclosure are capable of reducing an mRNA level in a cell, e.g., a SH-SY5Y cell, wherein the PRRT2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the KCNJ2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the TNK2 mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing an mRNA level in a cell, e.g., a SH-SY5Y cell, wherein the A TXN mRNA level is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

In some aspects, the oligonucleotides of the disclosure are capable of reducing a protein expression in a cell, e.g., a SH-SY5Y cell, wherein the protein comprises a V-type proton ATPase 116 kDa subunit a 2 (ATP6VOA2), a disintegrin and metalloproteinase domain-containing protein 10 (ADAM10), a protocadherin-19 (PCDH19), or any combination thereof.

In some aspects, the oligonucleotides of the disclosure are capable of reducing a ATP6VOA2 protein expression in a cell, e.g., a SH-SY5Y cell, wherein the ATP6VOA2 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing a ADAM10 protein expression in a cell, e.g., a SH-SY5Y cell, wherein the ADAM10 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide. In some aspects, the oligonucleotides of the disclosure are capable of reducing a PCDH19 protein expression in a cell, e.g., a SH-SY5Y cell, wherein the PCDH19 protein expression is reduced at least about one fold, at least two folds, at least three folds, at least four folds, at least five folds, at least six folds, at least seven folds, at least eight folds, at least nine folds, or at least ten folds compared to a cell that is not contacted with the oligonucleotide.

The present disclosure is directed to a method of reducing an occurrence, a duration, or a symptom of a seizure in a subject in need thereof comprising administering an oligonucleotide comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), e.g., any oligonucleotides disclosed herein.

The present disclosure is directed to a method of treating an epilepsy in a subject in need thereof comprising administering an oligonucleotide comprising a contiguous nucleotide sequence of 10 to 30 nucleic acids in length that are complementary to a nucleic acid sequence within miRNA-484 (5′-UCAGGCUCAGUCCCCUCCCGAU-3′ (SEQ ID NO: 1)), e.g., any oligonucleotides disclosed herein.

In some aspects, a miR-484 inhibitor disclosed herein can be administered by any suitable route known in the art. In certain aspects, a miR-484 inhibitor is administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracisternally, intracapsularly, intratumorally, or any combination thereof. In certain aspects, a miR-484 inhibitor is administered intracerebroventricularly (ICV). In certain aspects, a miR-484 inhibitor is administered intravenously.

In some aspects, a miR-484 inhibitor of the present disclosure can be used in combination with one or more additional therapeutic agents. In some aspects, the additional therapeutic agent and the miR-484 inhibitor are administered concurrently. In certain aspects, the additional therapeutic agent and the miR-484 inhibitor are administered sequentially.

In some aspects, the administration of a miR-484 inhibitor disclosed herein does not result in any adverse effects. In certain aspects, miR-484 inhibitors of the present disclosure do not adversely affect body weight when administered to a subject. In some aspects, miR-484 inhibitors disclosed herein do not result in increased mortality or cause pathological abnormalities when administered to a subject.

### VI. Kits

The present disclosure also provides kits or products of manufacture, comprising a miRNA inhibitor of the present disclosure (e.g., a polynucleotide, vector, or pharmaceutical composition disclosed herein) and optionally instructions for use, e.g., instructions for use according to the methods disclosed herein. In some aspects, the kit or product of manufacture comprises a miR-484 inhibitor (e.g., vector, e.g., an AAV vector, a polynucleotide, or a pharmaceutical composition of the present disclosure) in one or more containers. In some aspects, the kit or product of manufacture comprises miR-484 inhibitor (e.g., a vector, e.g., an AAV vector, a polynucleotide, or a pharmaceutical composition of the present disclosure) and a brochure. One skilled in the art will readily recognize that miR-484 inhibitors disclosed herein (e.g., vectors, polynucleotides, and pharmaceutical compositions of the present disclosure, or combinations thereof) can be readily incorporated into one of the established kit formats which are well known in the art.

The following examples are offered by way of illustration and not by way of limitation.

### EXAMPLES

### Example 1: Preparation of Oligonucleotides

The unmodified miR-484 ASO was chemically synthesized and consists of a 22-mer of RNA. All oligonucleotides were automatically synthesized by an oligo synthesizer (AKTA oligopilot10, GE Healthcare). In brief, ribo C-300 (133.78 mg, 40 μmol) as a Primer Support was filled into the column and the sequence information was entered into the AKTA software. The water contents, UV and pressure were monitored until a 22-mer oligomer was synthesized. After the reaction, the column was separated from the oligo synthesizer and washed with acetonitrile. The CpG was dried in a centrifuge evaporator (EYELA) and separated from oligonucleotide by the following steps. For the separation of CpG from the oligonucleotide, dried CpG was mixed with 4 mL of methylamine (40%) and an ethanol mixture (3:1, v/v) solution and stirred for 2 hours at 25° C. The mixture was filtered using a syringe filter (0.45 μm) and then dried in a centrifuge evaporator. The dried powder was dissolved into 1-methyl-2-pyrrolidone (NMP) (7680 l). Triethylamine (3840 l) and triethylamine tri-hydro fluoride (5120 l) were sequentially added into the solution. The mixture solution was maintained with stirring for 2 hrs at 65° C. 30 mL of isopropyl alcohol and 3 mL of 3M sodium acetate were sequentially added into the solution and stored at −20° C. for 12 hours for precipitation. The precipitate was separated by centrifuge (13,000 rpm, 30 min) and dried in a centrifuge evaporator.

The synthesized oligonucleotides were purified using Fast Protein Liquid Chromatography (FPLC) (AKTA Pure, GE Healthcare) attached with a Source tricon 10/100 GL column (GE Healthcare). As a mobile phase 10 mM of PB (phosphate buffer) and 1M of NaCl were used and the UV peaks of the oligonucleotides were detected at 260 nm wavelengths. After purifying the oligonucleotides, the remaining salt was removed by a desalting column (HiTrap Desalting, GE Healthcare). The solution was quantified using a Nanodrop (Thermo) and lyophilized, and white powder was obtained. The purity of final product was analyzed by HPLC (Agilent) and the molecular weight was measured by MALDI-TOF (Bruker). The total molecular weight of a free form of the ASO is 7175.4 g/mol.

### Example 2: In Vitro miR-484 Level Reduction

Neuroblastoma cell line SH-SY5Y was cultured in 1:1 mixture of Ham's F12 and Dulbecco's modified Eagle's medium (DMEM; Hyclone, Logan, UT, USA) with 10% fetal bovine serum (FBS; Hyclone, Logan, UT, USA) and 1% penicillin/streptomycin (Gibco, Carlsbad, CA, USA) for 3-4 days before experimentation and then sub-cultured for differentiation in 12 well culture plates (Corning Costar, Corning, NY, USA) at 1.5×105 cells/well.

A day after plating, media was removed and replaced SH-SY5Y media with 3% FBS containing 10 μM all-trans-Retinoic acid (ATRA, Sigma-Aldrich, St. Louis, MO, USA) and cells were grown for 3 days; then media was replaced SH-SY5Y media with 3% FBS containing 80 nM 12-O-tetradecanoyl-phorbol-13-acetate (TPA, Sigma-Aldrich, St. Louis, MO, USA) and cells were grown for 3 days. At the end of 6 days of differentiation, media was replaced SH-SY5Y media with 3% FBS. BV-2 cells were cultured in Dulbecco's modified Eagle's medium (DMEM; Hyclone, Logan, UT, USA) added with 10% FBS (Hyclone, Logan, UT, USA) and 1% penicillin/streptomycin (Gibco, Carlsbad, CA, USA) and sub-cultured every 2-3 days.

In order to detect the knockdown efficiency of the oligonucleotides, the SH-SY5 (2×10\hat{ }5 cells/well) and BV-2 (1.5×10≡cells/well) cells were cultured in DMEM supplemented with 5% FBS and 1× penicillin-streptomycin at 37° C., 5% CO₂ condition. Twelve-hours later, 500 nM of unmodified miR-484 ASO was transfected into the SH-SY5Y and BV-2 cells using Lipofectamine RNAiMAX (Invitrogen, Carlsbad, CA, USA) according to the manufacturer's protocol. The cells were incubated for 6, 12, and 24 hours at 37° C., 5% CO₂ condition. Cells were harvested and isolated RNA and then performed qRT-PCR.

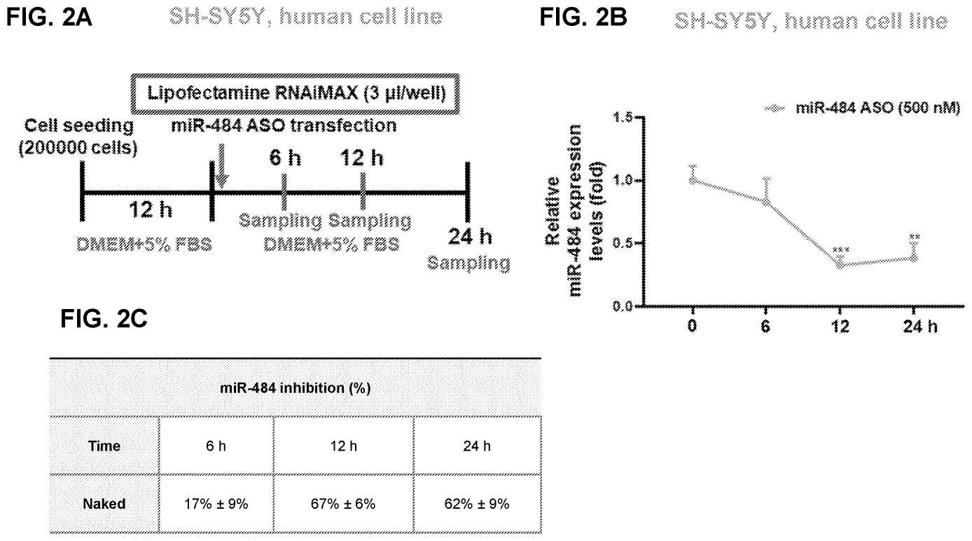

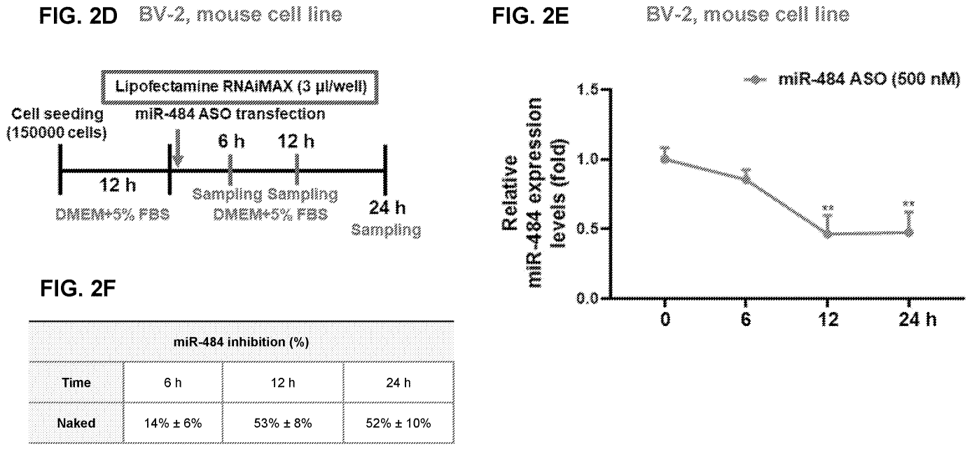

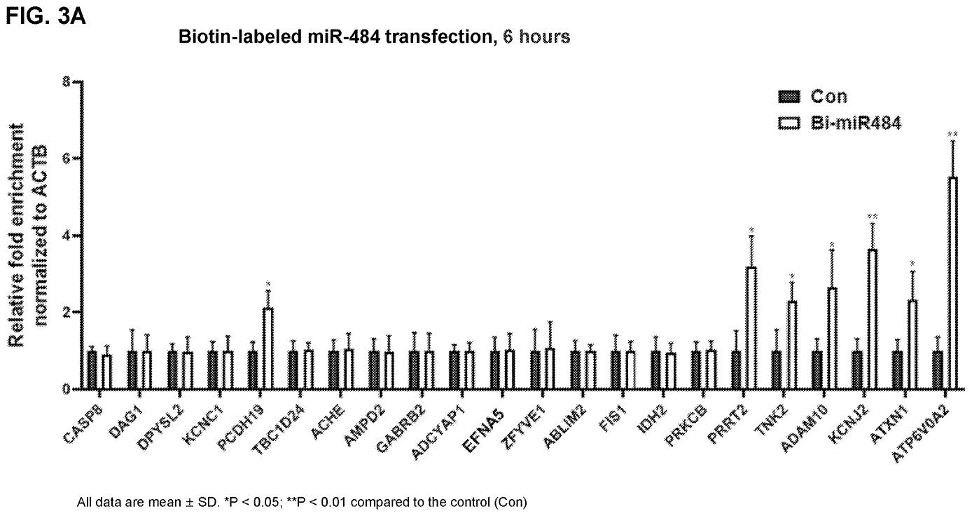

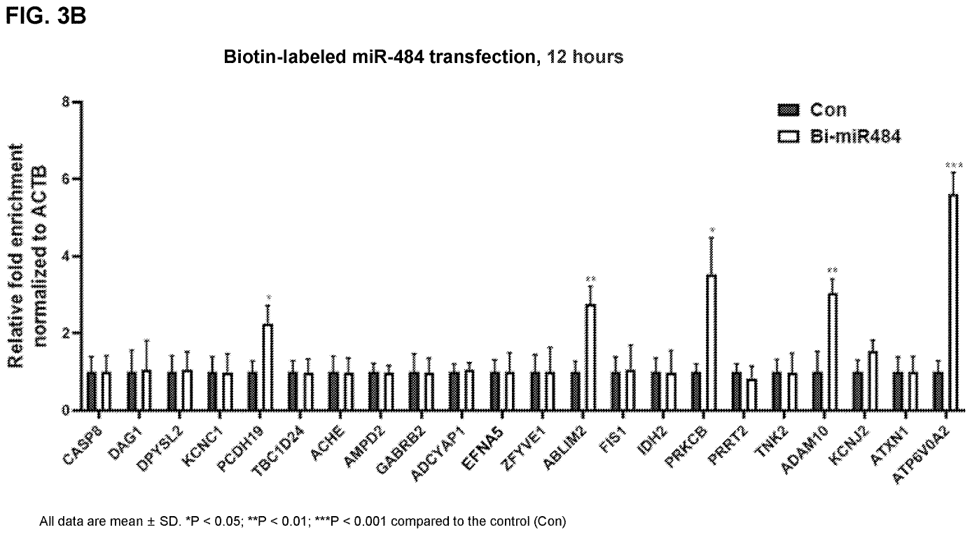

To test whether unmodified miR-484 ASO inhibits miR-484 expression, the unmodified miR-484 ASO was transfected into SH-SY5Y and BV-2 cell lines. See FIG. 2A and FIG. 2C. The data show the knockdown effect that the expression levels of miR-484 reduced at 6, 12, and 24 hours in SH-SY5Y (6h; 17±9%, 12h; 67±6%, 24h; 62±9%) and BV-2 cell lines (6h; 14±6%, 12h; 53±8%, 24h; 52±10%). See FIG. 2A and FIG. 2C. Unmodified ASO inhibits the miR-484 expression levels by up to 67% at 12 hours in SH-SY5Y cells and by 53% in BV-2 cells. FIG. 2B and FIG. 2D.

### Example 3: miR-484 Target Gene Modulation

To validate mRNA targets for miR-484, biotin-based pulldown assay was performed. The assay was performed as previously described (Lal A, Thomas MP, Altschuler G, Navarro F, O'Day E, et al. (2011) Capture of MicroRNA-Bound mRNAs identifies the Tumor Suppressor miR-34a as a Regulator of Growth Factor Signaling. PLoS Genet 7(11): e1002363, Dash et al., Biotin-based pulldown assay to validate mRNA targets of cellular miRNAs, J. Vis. Exp (2018) 136; 57786). The SH-SY5Y and BV-2 cells were cultured in DMEM supplemented with 10% FBS and 1× penicillin-streptomycin antibiotics at 37° C., 5% CO₂ condition. Biotin was attached to the 3′-end of the active strand in miR-484 and negative control. The biotinylated miR-484 mimic and negative control were transfected into the SH-SY5Y and BV-2 cells using Lipofectamine. The cells were incubated for 12, and 24 hours at 37° C., 5% CO₂ condition. Biotin-based pulldown was conducted with streptavidin coated magnetic beads. After 12 and 24 hours, the SH-SY5Y and BV-2 cells were harvested. Cell lysis was performed in 0.5 ml RIPA buffer (iNtRON Biotechnology, Seongnam, South Korea) with protease inhibitor cocktail (Roche, Mannheim, Germany) and RNase inhibitor (Enzynomics, Daejeon, South Korea). Streptavidin-coated magnetic beads (Invitrogen, Carlsbad, CA, USA) were blocked for 2 hours at 4° C. in lysis buffer containing 1 mg/ml yeast tRNA and 1 mg/ml BSA (Ambion, Austin, TX, USA) and washed twice with 1 ml lysis buffer. Cytoplasmic lysate was added to the beads and incubated for 4 h at 4° C. before the beads were washed five times with 1 ml lysis buffer. RNA bound to the beads (pull-down RNA) or from 10% of the extract (input RNA), was isolated using QIAzol (QIAGEN Sciences, Maryland, MD, USA).

We used SH-SYSY and BV-2 cells to study binding partners between miR-484 and 22 candidate target genes. The candidate target genes of miR-484 were selected regulating epileptic condition as shown in the following table:

TABLE 1
qRT-PCR

| | | | |
|---|---|---|---|
| 1 | Casp8 | F | AGAAGAGGGTCATCCTGGGAGA (seq id no: 161) |
| | | R | CCCGAGGTTTGCTTTTCATTTG (seq id no: 74) |
| 2 | Dag1 | F | CCACAGTGGTTGGCATTCCTGA (seq id no: 75) |
| | | R | CCACAGTGGTTGGCATTCCTGA (seq id no: 76) |
| 3 | Dpysl2 | F | GCCAGATTTATGAAGTACTGAGTG (seq id no: 77) |
| | | R | GGCGATGACCTCAGCAGAGCTT (seq id no: 78) |
| 4 | Kcnc1 | F | GCTGAGCGGCCTGTCCTCCAAG (seq id no: 79) |
| | | R | GCCCGCCAGCGCACACAGAGCC (seq id no: 80) |
| 5 | Pcdh19 | F | CGACCACCCGGCTGAGGAGAGG (seq id no: 81) |
| | | R | GTTGACATACTGCTCCAGATCAC (seq id no: 82) |
| 6 | Tbc1d24 | F | CACGTTCGTCAGAGACATCGCG (seq id no: 83) |
| | | R | CTCACGCTGACGATCTCCGAGC (seq id no: 84) |
| 7 | Zfyve1 | F | GCCATAAAAGACCCTTCCAAGTC (seq id no: 85) |
| | | R | GAGTCTGAAGACTGCACGAAACTTC (seq id no: 86) |
| 8 | Ache | F | CATGGGACCCCGTCGCTTTCTG (seq id no: 87) |
| | | R | CTGTACCAAGAAGCGGCCATCG (seq id no: 88) |
| 9 | Ampd2 | F | GTGAACAGACGCTGGGGGAGGT (seq id no: 89) |
| | | R | GATACCCTGTTGTCCGTCTTGATG (seq id no: 90) |
| 10 | Adcyap1 | F | GATCCTTAACGAGGCCTACCGC (seq id no: 91) |
| | | R | GTAACCCATCGCTACAAATAAGC (seq id no: 92) |
| 11 | Gabrb2 | F | CTGAGGCTGTGATGGGACTTGG (seq id no: 93) |
| | | R | CAGTGGGAGGCCATGTTTTAG (seq id no: 94) |
| 12 | Efna5 | F | CAGACTTCGTGAATGGGAATTTG (seq id no: 95) |
| | | R | CAGAGGAGTTCCTTATGTCAGAG (seq id no: 96) |
| 13 | Ablim2 | F | CGGATGGGGAGGATGGAAGCTT (seq id no: 97) |
| | | R | GATTGGCATTCCGCTGGTCCAA (seq id no: 98) |
| 14 | Fis1 | F | GCGGAGCAAGTACAATGATGACA (seq id no: 99) |
| | | R | CAATGAGCCGCTCCAGTTCCTT (seq id no: 100) |
| 15 | Tdh2 | F | GTGGTGTCAAGGAGTGGGAAG (seq id no: 101) |
| | | R | CATCAATGAGCCGGTGCTCAT (seq id no: 102) |
| 16 | Prkcb | F | GTCACTGCTGTATGGACTCATC (seq id no: 103) |
| | | R | GAGGACAATGAGGACGTCCCTG (seq id no: 104) |
| 17 | Prrt2 | F | GTTGAGGAGAGTCCCAAGGTT (seq id no: 105) |
| | | R | GATTCCCCTCCTGGGCTTAAGC (seq id no: 106) |
| 18 | Tnk2 | F | GTCCCACTTTGAGTACGTCAAGA (seq id no: 107) |
| | | R | GGTGGGAACTCAGCCTCCAGT (seq id no: 108) |
| 19 | Adam10 | F | CAAGTCAAGGTCCTTGTTGTACA (seq id no: 109) |
| | | R | CGTACACTCCTCTAAGCCATAT (seq id no: 110) |
| 20 | Kcnj2 | F | CGAGAGGCAGAGACTATGCGA (seq id no: 111) |
| | | R | GAGTIGTCCAAATTCGTCCGTT (seq id no: 112) |
| 21 | Atxn1 | F | CGTCATGCAATACGCCGACTC (seq id no: 113) |
| | | R | GTACGGGTGAGGAACCGACTTG (seq id no: 114) |
| 22 | Atp6v0a2 | F | CCAAGGAAAAGTGGAAGCATTTG (seq id no: 115) |
| | | R | GGCTGTGTTTGGATAGGGGTAC (seq id no: 116) |

qRT-PCR

| No | Gene | 5′-primer sequence-3′ (Mouse) | |
|---|---|---|---|
| 1 | Casp8 | F | GTATCTTACGACGACTGCACTG (seq id no: 117) |
| | | R | CTTCCTTGGCAAGCCTGAATG (seq id no: 118) |
| 2 | Dag1 | F | GCCAGTGACTGTCCTTACAGTG (seq id no: 119) |
| | | R | GTTTCTACACCACGGATGTCAG (seq id no: 120) |
| 3 | Dpysl2 | F | CTGAAGCTGTGAACCGGTCC (seq id no: 121) |
| | | R | GAGAGCCATCAGTCCCCAGGC (seq id no: 122) |
| 4 | Kcnc1 | F | GCAGTCAGTCATCGGTCACA (seq id no: 123) |
| | | R | GACAGAGGTGGAAGAGGGTG (seq id no: 124) |
| 5 | Pcdh19 | F | GCCATCTGGGTTCGAGGAAG (seq id no: 125) |
| | | R | CGAGAGGGATTGTCACAGAATG (seq id no: 126) |
| 6 | Tbc1d24 | F | CTGCAGGCAGTTCTTCCCTC (seq id no: 127) |
| | | R | GTCCAGTGCGCCCTCGATTC (seq id no: 128) |
| 7 | Zfyve1 | F | CTAGAGCAGCCCGGGTCCT (seq id no: 129) |
| | | R | GCAGCAGTGTCCACTGAATT (seq id no: 130) |
| 8 | Ache | F | CTGGTATCCCCTGCATACACC (seq id no: 131) |
| | | R | CATCCAACACTCCTGACCAGG (seq id no: 132) |
| 9 | Ampd2 | F | GTATTGGAACGGGAATTTCAGC (seq id no: 133) |
| | | R | CAGGACTCTGCTCATAAGTTCG (seq id no: 134) |
| 10 | Adcyap1 | F | GAGAGCAATTAAGATGGGTGCA (seq id no: 135) |
| | | R | CTTGAGTTAGTTAGGCTGGGGTT (seq id no: 136) |
| 11 | Gabrb2 | F | CTACAGCCTTCCGTGAGAAGC (seq id no: 137) |
| | | R | CACACTTGATCTTGCCAGCATG (seq id no: 138) |
| 12 | Efna5 | F | GAATGAACATGGGATGACCCTG (seq id no: 139) |
| | | R | CACTTTTCCATTGGTCTGACACTA (seq id no: 140) |
| 13 | Ablim2 | F | GACCTCCTTCCTCTACCTACC (seq id no: 141) |
| | | R | GTTCCTGGTCAGTCCCGCTG (seq id no: 142) |
| 14 | Fis1 | F | GACCTTAGTGTGAGGCTTTCAGG (seq id no: 143) |
| | | R | CAGGCGTTCCAGCTCCTTG (seq id no: 144) |
| 15 | Tdh2 | F | CATTACCGAGAACACCAGAAGG (seq id no: 145) |
| | | R | GGAGCTGAGTAGTGTCCACC (seq id no: 146) |
| 16 | Prkcb | F | CTCTATTGGCATCCGTCATGTG (seq id no: 147) |
| | | R | CCGGTCATGCTTAGGTAGCA (seq id no: 148) |
| 17 | Prrt2 | F | CCAGGCATTCTGAAGAAGGAC (seq id no: 149) |
| | | R | CTCAGACCCTTCCTCTGCTGC (seq id no: 150) |
| 18 | Tnk2 | F | GCCCTCAAGACATCTACAATGT (seq id no: 151) |
| | | R | CGACATTTCGAGGGAAGGGTC (seq id no: 152) |
| 19 | Adam10 | F | GTGAGCAGGAGAGGCTGTG (seq id no: 153) |
| | | R | CAACTACCTGCTGAGCAAATCAC (seq id no: 154) |
| 20 | Kcnj2 | F | CAATGCGGCTTCTGTGAGCC (seq id no: 155) |
| | | R | CATCAGCTAGCTCCCACGGA (seq id no: 156) |
| 21 | Atxn1 | F | GTGGTTGTGCAATATAGTGATGCC (seq id no: 157) |
| | | R | GAGGGTGTGCTGCTATTAGGCA (seq id no: 158) |
| 22 | Atp6v0a2 | F | GTTGGTGGCTCTCACGGTGC (seq id no: 159) |
| | | R | GCCTGCGTCATCAGGATCTC (seq id no: 160) |

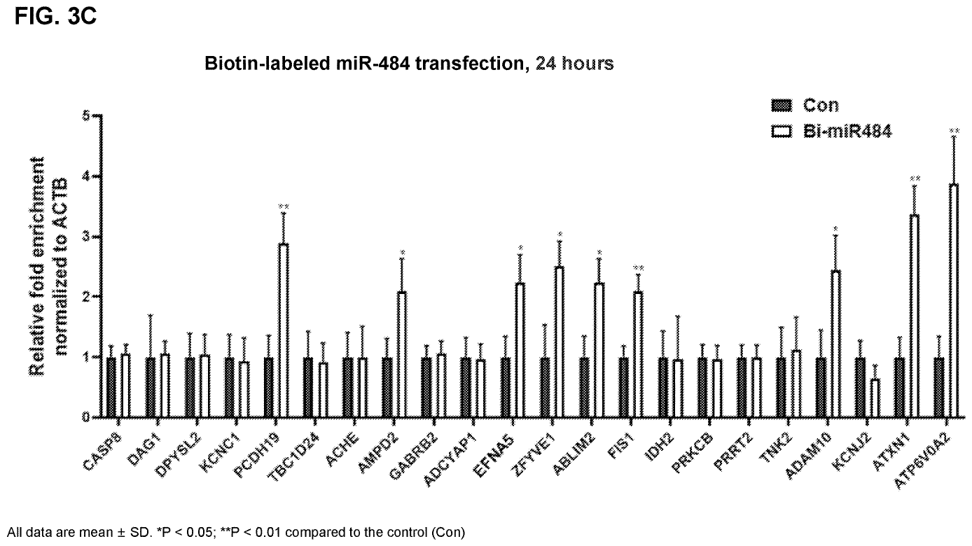

The expression of 11 of 22 candidate genes binding to miR-484 were changed in both SH-SY5Y and BV-2 cells. Expression of some of the candidate genes was increased in a time-dependent manner. Expression of a few candidate genes was increased shortly after the contact with miR-484 mimic and then degraded. The number of candidate genes were shown increase the expression levels by binding of miR-484 mimic according to 6, 12, and 24 hours after biotinylated miR-484 mimic in SH-SY5Y cells. After 6 hours of transfection in SH-SY5Y, the following-genes indicate increased expression level by binding to the biotinylated miR-484 mimic: Table 2. See also FIG. 3A.

TABLE 2
See also FIG. 3A.
Relative fold change

| | | |
|---|---|---|
| 1 | ATP6V0A2 | 6.11 ± 1.9 |
| 2 | PRRT2 | 4.32 ± 2.8 |
| 3 | KCNJ2 | 4.07 ± 1.6 |
| 4 | TNK2 | 2.85 ± 1.3 |
| 5 | ADAM10 | 2.61 ± 0.2 |
| 6 | ATXN1 | 2.32 ± 0.2 |
| 7 | PCDH19 | 2.19 ± 0.5 |

After 12 hours of transfection in SH-SY5Y, the following-genes indicate increased expression level by binding to the biotinylated miR-484 mimic:

TABLE 3
See also FIG. 3B.
Relative fold change

| | | |
|---|---|---|
| 1 | ATP6V0A2 | 5.82 ± 0.85 |
| 2 | ADAM10 | 4.08 ± 2.42 |
| 3 | PRKCB | 3.74 ± 1.22 |
| 4 | PCDH19 | 3.08 ± 0.5 |
| 5 | ABLIM2 | 2.85 ± 0.48 |

After 24 hours of transfection in SH-SYSY, the following-genes indicate increased expression level by binding to the biotinylated miR-484 mimic: Table 4. See also FIG. 3C.

TABLE 4
See also FIG. 3C.
Relative fold change

| | | |
|---|---|---|
| 1 | ATP6V0A2 | 4.3 ± 0.63 |
| 2 | ATXN1 | 3.78 ± 0.38 |
| 3 | PCDH19 | 3.19 ± 1.18 |
| 4 | ZFYVE1 | 2.9 ± 0.97 |
| 5 | ADAM10 | 2.74 ± 0.47 |
| 6 | EFNA5 | 2.41 ± 0.81 |
| 7 | ABLIM2 | 2.39 ± 0.97 |
| 8 | Fis1 | 2.14 ± 0.48 |
| 9 | AMPD2 | 2.11 ± 0.09 |

After 12 hours of transfection in BV-2 cell line, the following-genes indicate increased expression level by binding to the biotinylated miR-484 mimic:

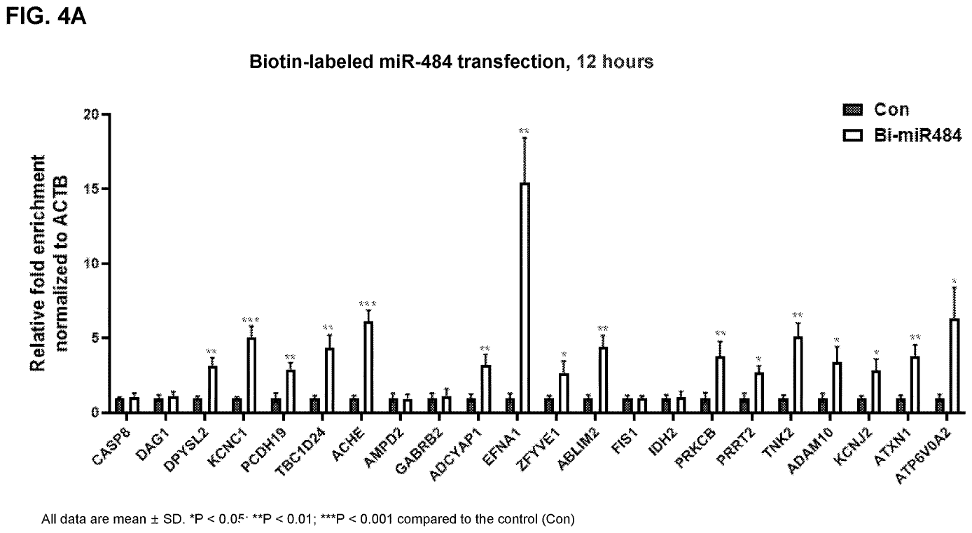

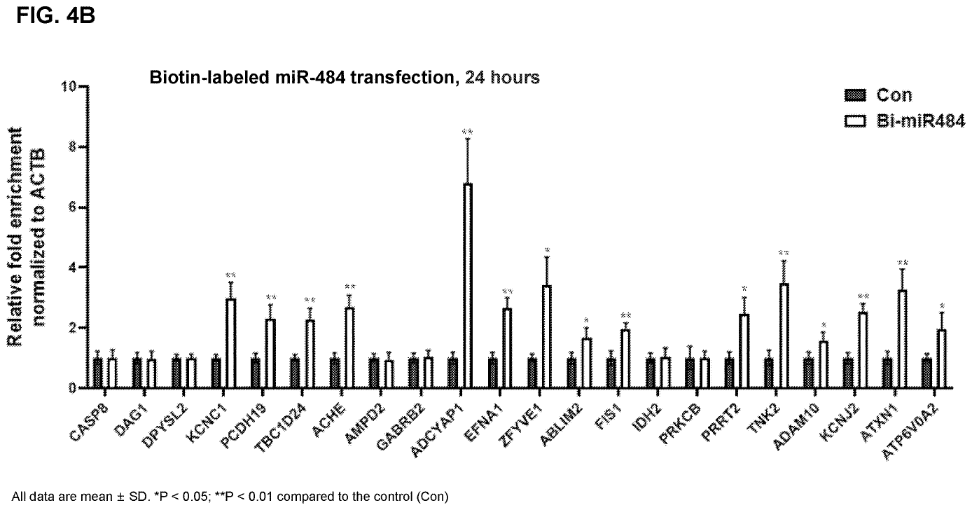

TABLE 5
See Also FIG. 4A.
Relative Fold Change

| | | |
|---|---|---|
| 1 | EFNA5 | 17.29 ± 7.55 |
| 2 | ATP6V0A2 | 6.93 ± 3.13 |
| 3 | ACHE | 6.16 ± 0.49 |
| 4 | TNK2 | 5.26 ± 1.11 |
| 5 | KCNC1 | 5.08 ± 0.86 |
| 6 | ABLIM2 | 4.49 ± 0.71 |
| 7 | TBC1D24 | 4.41 ± 0.97 |
| 8 | KCNJ2 | 3.88 ± 0.81 |
| 9 | PRKCB | 3.86 ± 0.29 |
| 10 | ADAM10 | 3.62 ± 1.21 |
| 11 | ADCYAP1 | 3.45 ± 1.29 |
| 12 | DPYSL2 | 3.16 ± 0.61 |
| 13 | PCDH19 | 3.01 ± 0.41 |
| 14 | ATXN1 | 2.91 ± 0.86 |
| 15 | PRRT2 | 2.75 ± 0.27 |
| 16 | ZFYVE1 | 2.69 ± 0.72 |

After 24 hours of transfection in BV-2 cell line, the following-genes indicate increased expression level by binding to the biotinylated miR-484 mimic:

TABLE 6
(See Also FIG. 4B)
Relative fold increase

| | | |
|---|---|---|
| 1 | ADCYPA1 | 6.78 ± 0.31 |
| 2 | TNK2 | 3.76 ± 1.33 |
| 3 | ATXN1 | 3.38 ± 0.92 |
| 4 | ZFYVE1 | 3.36 ± 0.39 |
| 5 | KCNC1 | 3.03 ± 0.71 |
| 6 | ACHE | 2.73 ± 0.39 |
| 7 | EFNA5 | 2.67 ± 0.13 |
| 8 | KCNJ2 | 2.59 ± 0.43 |
| 9 | PRRT2 | 2.47 ± 0.05 |
| 10 | PCDH19 | 2.32 ± 0.33 |
| 11 | TBC1D24 | 2.31 ± 0.52 |
| 12 | Fis1 | 2.05 ± 0.46 |
| 13 | ATP6V0A2 | 1.95 ± 0.35 |
| 14 | ABLIM2 | 1.74 ± 0.54 |
| 15 | ADAM10 | 1.65 ± 0.52 |

### Example 4: In vivo Efficacy in mTLE Model

Animals for epilepsy model were prepared as follows. Male C57BL/6 mice were obtained from Orient Bio, Inc (Seongnam-S1, Gyeonggi-Do, Korea) and used for pilocarpine-induced SRS model and kainic acid induced mTLE (mesial temporal lobe epilepsy) model. Animals were housed under controlled conditions. Conditions of the animal room during the study conformed to the following; temperature was maintained at 19 to 25° C. and humidity was maintained at 30 to 70%. 12 hours of light/dark cycle each day and air ventilation was 10 to 20 times an hour. Pelleted food and tap water were provided ad libitum.

Kainic acid (KA) induced mTLE model (mTLE model) was generated as follows: Male C57BL/6 mice (8 weeks age, 18~24 g) were euthanized with isoflurane and placed on a stereotaxic and set the brain position. Micro-syringe needle was inserted to the target point (AP −2.0, ML −1.5, DV −2.0). KA solution or nuclease free water (flow rate 0.5 l/min) was injected and 3 minutes elapsed before withdrawal. Only mice showing epileptic behaviors were selected and administrated with 5 mg/kg of diazepam 1 hour after KA injection to reduce mortality and status epilepticus (SE). About 4 weeks after KA injection, KA-induced mice were injected intracerebroventricularly with ASO and implanted with an EEG socket. 7 mice intracerebroventricular (icv) single injection with nuclease free water as a vehicle group at a volume of 4 μL. 8 mice intracerebroventricular (icv) single injection with tested article at a dose of 2 g in 4 μL. About 5 weeks after KA injection, seizure recording (EEG recording) was performed to detect seizure activity from hippocampal regions at least five hours. Seizure count and duration were analyzed with NeuroScore Version 3.0 program (DSI, USA) and their statistical differences are analyzed and graphically displayed with GraphPad (Prism 7.0).

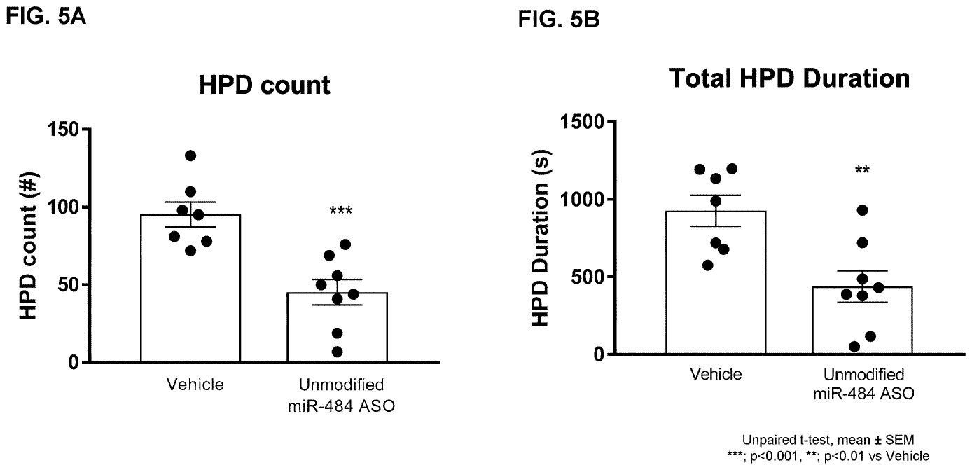

To measure electrophysiological changes of seizure activity after KA injection, EEG recordings from hippocampus were performed. The HPD (hippocampal paroxysmal discharges) in KA mTLE mouse model were shown to be significantly reduced by administration of unmodified miR-484 ASO. Silencing of miR-484 which is upregulated in mTLE mouse model, by unmodified miR-484 ASO significantly reduced both HPD count (52.5%) and duration (52.8%) in chronic epilepsy models compared to vehicle group. FIGS. 5A and 5B, respectively.

### Example 5: In vivo Efficacy in Pilocarpine Induced SRS Model

To conduct 6 Hz test with pilocarpine induced SRS (spontaneous recurrent seizure) model, male C₅₇BL/6 mice (7 weeks age, 19-22 g) were used to pilocarpine-induced SRS model. Methylscopolamine (1 mg/kg, 10 ml/kg) was dissolved in a 0.9% saline and administered subcutaneously to the back of the neck. After 30 minutes, pilocarpine (340 mg/kg, 10 ml/kg) was dissolved in a 0.9% saline and administered intraperitoneally. When animals showed continues clonic seizures, after 40 min, diazepam (5 mg/kg, 10 ml/kg) was injected for cessation of status epilepticus (SE). At least 4 weeks after SE induction (latent period), animals were randomly assigned to experimental groups. 20 mice intracerebroventricular (icv) single injection with nuclease free water as a vehicle group at a volume of 4 μL. 34 mice intracerebroventricular (icv) single injection with tested article at a dose of 2 g in 4 μL. 6 Hz electroshock (0.2 ms rectangular pulse width, 3 sec duration) was introduced onto the eyes of mice using corneal electrode linked to the shock generator. Test was performed through up-and-down method. If animal show seizure, decrease current and if not, increase current for next animal. Seizure behaviors (Stun, forelimb clonus, twitching of vibrissae, straub-tail, mouth chomping, etc.) or seizure protection were recorded.

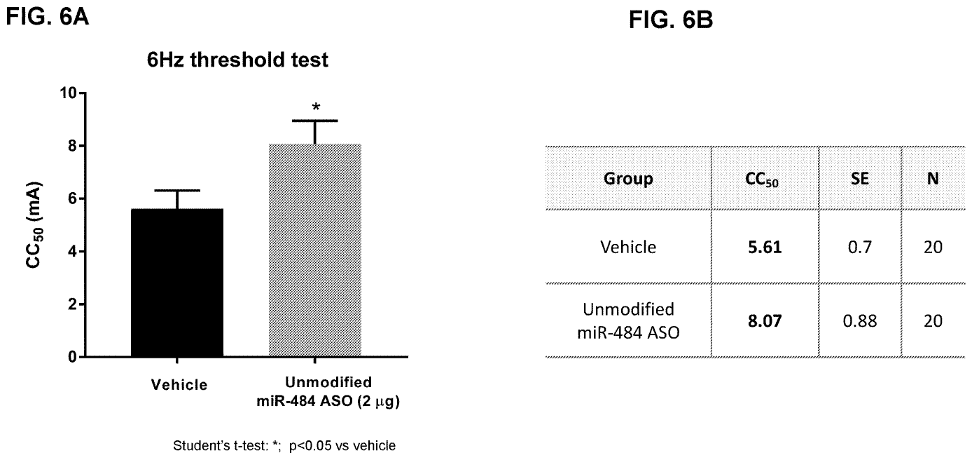

The CC₅₀ was determined in the 6-Hz test and compared to that of vehicle group in pilocarpine-induced SRS mice. Unmodified miR-484 ASO increased CC₅₀ value of pilocarpine-induced SRS model compared to that of vehicle group. As shown in FIGS. 6A and 6B.

In addition, the knockdown efficiency of unmodified miR-484 ASO was determined by qRT-PCR in brain tissues in vehicle and pilocarpine-induced SRS mice model. To isolate RNA for quantitative RT-PCR, total RNA was isolated using the small and large RNA kit (Macherey-Nagel, Allentown, PA, USA) and QIAzol lysis reagent (QIAGEN Sciences, Maryland, MD, USA). miScript II RT Kit (Qiagen) was used for cDNA synthesis for miRNA detection and quantification. The reverse-transcribed product was subjected to qRT-PCR using TAQMAN™ miRNA assay (ThermoFisher Scientific, Waltham, MA, USA) and primers (Table 1). TOPSCRIPT™ RT DryMIX (Enzynomics, Daej eon, Korea) was used for cDNA synthesis for mRNA detection and quantification. The reverse-transcribed product was subjected to qRT-PCR using TOPREAL™ qPCR 2×PreMIX, SYBR Green (Enzynomics, Daejeon, South Korea), and primers (Table 1). PCR primers were commercially synthesized (Bioneer, Daejeon, South Korea). A 50-cycle amplification was applied for all primers using the CFX96 Real-Time System (Bio-Rad, Hercules, CA, USA). The normalization was performed using ACTB as the reference gene.

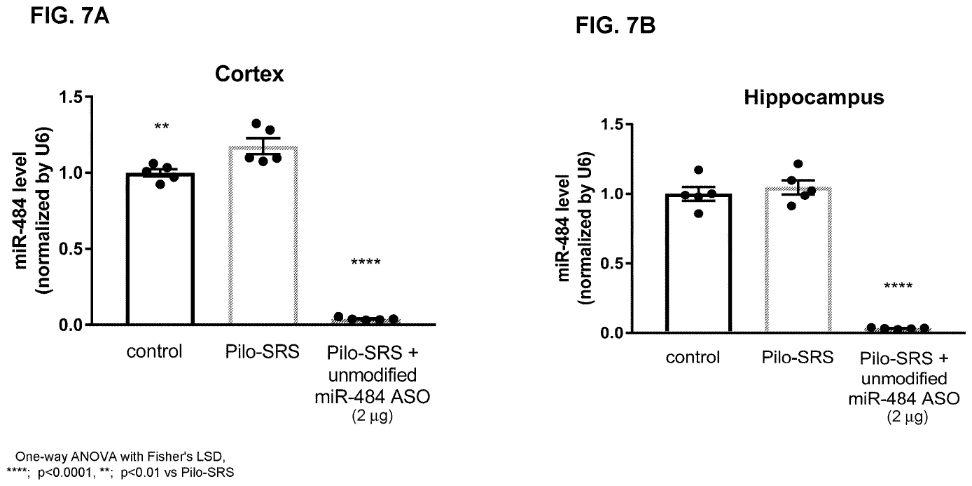

The expression levels of miR-484 were significantly decreased in both cortex and hippocampus of pilocarpine-induced SRS model at 1-week post ICV injection. FIGS. 7A and 7B.

### Example 6: Preparation of Modified Oligonucleotides

For the synthesis of modified oligonucleotides: each RNA amidite (A, U, G, C) was purchased from Hongene Biotech. Locked Nucleic Acid (LNA) Phosphoramidite (A, T, G, C) and 2′-O-methoxyethyl (2′-MOE) Phosphoramidite was purchased from Glen research. As a Unylinker Support for oligonucleotide synthesis, CpG was purchased from GE healthcare. Acetonitrile (synthesis quality, water content <25 ppm) and Toluene was supplied from Acros. All other reagents for RNA synthesis such as Detritylation, Activator, Capping A, Capping B1, Capping B2 were purchased from Sigma-Aldrich. To synthesize the phosphorothioate backbone, Sulfurizing Reagent II (Glen research) was used. The amidites and acetonitrile were dehydrated using a molecular sieve and incubated for 1 day (Thermo-Fisher). 25~28% Ammonium hydroxide for deprotection was purchased from Duksan. 1-methyl-2-pyrrolidone (NMP) was purchased from Daejung Chemical and NMP was distillated prior to use. Triethylamine and triethylamine tri-hydro fluoride were purchased from Sigma-Aldrich.

All oligonucleotides were automatically synthesized by oligo synthesizer (AKTA oligopilotl0, GE Healthcare). In brief, Primer Support 5G Unylinker 350 (58.14 mg, 20 μmol) was filled into the column and the sequence information was entered into the AKTA software. The water contents, UV and pressure were monitored until a 22-mer of oligomer was synthesized. After the reaction, the column was separated from oligo synthesizer and washed with acetonitrile. The CpG was dried in centrifuge evaporator (EYELA) and separated from the oligonucleotides by the following steps. For the separation of CpG from the oligonucleotides, dried CpG was mixed with 4 mL of ammonium hydroxide (25-28%) solution and stirred for 2 hours at 65° C. The mixture was filtered using a syringe filter (0.45 μm) and then dried in a centrifuge evaporator. The dried power was dissolved into 5400 μl of NMP, and then 2700 μl of triethylamine and 3600 μl of triethylamine tri-hydro fluoride were sequentially added into the solution. The mixture solution was maintained with stirring for 2 hours at 65° C. 25 mL of isopropyl alcohol and 1.5 mL of 3M sodium acetate were sequentially added into the solution and stored at −20° C. for 12 hours for precipitation. The precipitate was separated by centrifuge (13,000 rpm, 30 min) and dried in a centrifuge evaporator. The synthesized oligonucleotides were purified using high-performance liquid chromatography (HPLC) (Thermo ultimate 3000) attached with PLRP-S 300A column (Agilent Technologies). As a mobile phase 100 mM of TEAA in water and 100 mM TEAA in ACN were used and the UV peaks of the oligonucleotides were detected at 260 nm wavelength. After purifying the oligonucleotides, the remaining salt was removed by a desalting column (HiTrap Desalting, GE Healthcare). The solution was quantified using Nanodrop (Thermo) and lyophilized, and white powder was obtained. The final modified ASO comprises 5′-AUCGGGAGGGGACUGAGCCUGA-3′ (SEQ ID NO: 73), wherein the bolded letters are 2′-MOEs and the double underlined letters are 5-methyl pyrimidines and the backbone of the oligonucleotide is PS modified (miR-484 ASO M4). The purity of the final product was analyzed by HPLC (Agilent) and molecular weight was measured by MALDI-TOF (Bruker).

### Example 7: In Vivo Efficacy of Modified miR-484 ASO in the mTLE Model

Animal care, model generation, and data analysis for in vivo efficacy in the mTLE model were conducted as described in Example 4. About 4 weeks after KA (kainic acid) injection, KA-induced mice were injected intracerebroventricularly with EEG (electroencephalogram) socket implantations. The vehicle group was represented by five mice receiving a single intracerebroventricular (icv) injection with nuclease free water at a volume of 4 μL. The tested group was represented by nine mice receiving a single icv injection with a modified miR-484 ASO M4 at a dose of 1 g in 4 L of nuclease free water. About 5 weeks after KA injection, seizure recording (EEG recording) was performed to detect seizure activity from the hippocampal regions of the mice for at least five hours.

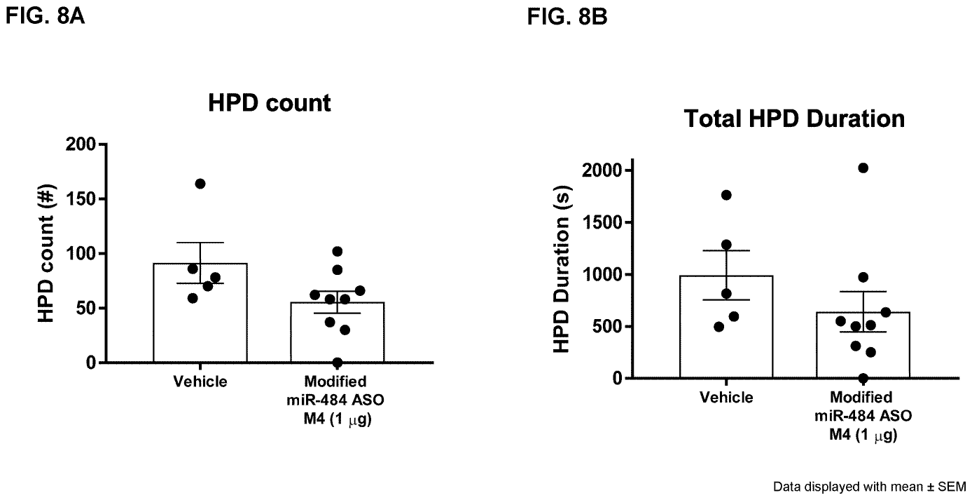

EEG recordings from the hippocampus were collected. The HPDs (hippocampal paroxysmal discharges) in the KA-induced mTLE mouse model were shown to be reduced by the injection of modified miR-484 ASO as prepared with the methodology of Example 6. Silencing of miR-484 by the modified miR-484 ASO reduced both HPD count (39.5%) and duration (35.5%) in chronic epilepsy models compared to vehicle group. The results of this study are shown in FIGS. 8A and 8B, respectively.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. An oligonucleotide comprising a contiguous nucleotide sequence of 10 to 30 nucleic acids in length that are complementary to a nucleic acid sequence within miRNA 484 (5′-UCAGGCUCAGUCCCCUCCCGAU-3′ (SEQ ID NO: 1)), wherein the contiguous nucleotide sequence comprises at least one nucleic acids analog.

2. The oligonucleotide of claim 1, wherein the at least one nucleic acid analog is a modified nucleic acid at the sugar position.

3. The oligonucleotide of claim 1 or 2, wherein the at least one nucleic acid analog is selected from Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleic acid (cEt), 2′-O-methyl nucleic acid (2′-OMe), 2′-O-methoxyethyl nucleic acid (2′-MOE), and any combination thereof.

4. The oligonucleotide of claim 1 or 2, wherein the at least one nucleic acid analog is a nucleic acid comprising a bicyclic sugar.

5. The oligonucleotide of claim 4, wherein the nucleic acid comprising a bicyclic sugar comprises cEt, 2′,4′-constrained 2′-O-methoxyethyl (cMOE), LNA, α-L-LNA, β-D-LNA, 2′-O,4′-C-ethylene-bridged nucleic acids (ENA), amino-LNA, oxy-LNA, or thio-LNA.

6. The oligonucleotide of any one of claims 1 to 5, wherein the at least one nucleic acid analog is 2′-OMe.

7. The oligonucleotide of any one of claims 1 to 6, wherein the contiguous nucleotide sequence comprises 5′-GAGCCUG-3′.

8. The oligonucleotide of any one of claims 1 to 7, wherein the oligonucleotide further comprises at least 1 nucleic acid, at least 2 nucleic acids, at least 3 nucleic acids, at least 4 nucleic acids, at least 5 nucleic acids, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 5′ of the contiguous nucleotide sequence.

9. The oligonucleotide of any one of claims 1 to 8, wherein the oligonucleotide further comprises at least 1 nucleic acid, at least 2 nucleic acids, at least 3 nucleic acids, at least 4 nucleic acids, at least 5 nucleic acids, at least 6 nucleic acids, at least 7 nucleic acids, at least 8 nucleic acids, at least 9 nucleic acids, at least 10 nucleic acids, at least 11 nucleic acids, at least 12 nucleic acids, at least 13 nucleic acids, at least 14 nucleic acids, at least 15 nucleic acids, at least 16 nucleic acids, at least 17 nucleic acids, at least 18 nucleic acids, at least 19 nucleic acids, or at least 20 nucleic acids at the 3′ of the contiguous nucleotide sequence.

10. The oligonucleotide of any one of claims 1 to 9, wherein the oligonucleotide has the sequence selected from the group consisting of: 5′-GAGCCUG-3′, 5′-GAGCCUGA-3′, 5′-UGAGCCUG-3′, 5′-CUGAGCCUG-3′, 5′-ACUGAGCCUG-3′ (SEQ ID NO: 6), 5′-GACUGAGCCUG-3′ (SEQ ID NO: 7), 5′-GGACUGAGCCUG-3′ (SEQ ID NO: 8), 5′-GGGACUGAGCCUG-3′ (SEQ ID NO: 9), 5′-GGGGACUGAGCCUG-3′ (SEQ ID NO: 10), 5′-AGGGGACUGAGCCUG-3′ (SEQ ID NO: 11), 5′-GAGGGGACUGAGCCUG-3′ (SEQ ID NO: 12), 5′-GGAGGGGACUGAGCCUG-3′ (SEQ ID NO: 13), 5′-GGGAGGGGACUGAGCCUG-3′ (SEQ ID NO: 14), 5′-CGGGAGGGGACUGAGCCUG-3′ (SEQ ID NO: 15); 5′-UCGGGAGGGGACUGAGCCUG-3′ (SEQ ID NO: 16), 5′-AUCGGGAGGGGACUGAGCCUG-3′ (SEQ ID NO: 17), 5′-UGAGCCUGA-3′, 5′-CUGAGCCUGA-3′ (SEQ ID NO: 19), 5′-ACUGAGCCUGA-3′ (SEQ ID NO: 20), 5′-GACUGAGCCUGA-3′ (SEQ ID NO: 21), 5′-GGACUGAGCCUGA-3′ (SEQ ID NO: 22), 5′-GGGACUGAGCCUGA-3′ (SEQ ID NO: 23), 5′-GGGGACUGAGCCUGA-3′ (SEQ ID NO: 24), 5′-AGGGGACUGAGCCUGA-3′ (SEQ ID NO: 25), 5′-GAGGGGACUGAGCCUGA-3′ (SEQ ID NO: 26), 5′-GGAGGGGACUGAGCCUGA-3′ (SEQ ID NO: 27), 5′-GGGAGGGGACUGAGCCUGA-3′ (SEQ ID NO: 28), 5′-CGGGAGGGGACUGAGCCUGA-3′ (SEQ ID NO: 29), 5′-UCGGGAGGGGACUGAGCCUGA-3′ (SEQ ID NO: 30), and 5′-AUCGGGAGGGGACUGAGCCUGA-3′ (SEQ ID NO: 31).

11. The oligonucleotide of any one of claims 1 to 10, wherein the oligonucleotide has the selected from the group consisting of: 5′-GAGCCTG-3′, 5′-GAGCCTGA-3′, 5′-TGAGCCTG-3′, 5′-CTGAGCCTG-3′, 5′-ACTGAGCCTG-3′ (SEQ ID NO: 36), 5′-GACTGAGCCTG-3′ (SEQ ID NO: 37), 5′-GGACTGAGCCTG-3′ (SEQ ID NO: 38), 5′-GGGACTGAGCCTG-3′ (SEQ ID NO: 39), 5′-GGGGACTGAGCCTG-3′ (SEQ ID NO: 40), 5′-AGGGGACTGAGCCTG-3′ (SEQ ID NO: 41), 5′-GAGGGGACTGAGCCTG-3′ (SEQ ID NO: 42), 5′-GGAGGGGACTGAGCCTG-3′ (SEQ ID NO: 43), 5′-GGGAGGGGACTGAGCCTG-3′ (SEQ ID NO: 44), 5′-CGGGAGGGGACTGAGCCTG-3′ (SEQ ID NO: 45); 5′-TCGGGAGGGGACTGAGCCTG-3′ (SEQ ID NO: 46), 5′-ATCGGGAGGGGACTGAGCCTG-3′ (SEQ ID NO: 47), 5′-TGAGCCTGA-3′, 5′-CTGAGCCTGA-3′ (SEQ ID NO: 49), 5′-ACTGAGCCTGA-3′ (SEQ ID NO: 50), 5′-GACTGAGCCTGA-3′ (SEQ ID NO: 51), 5′-GGACTGAGCCTGA-3′ (SEQ ID NO: 52), 5′-GGGACTGAGCCTGA-3′ (SEQ ID NO: 53), 5′-GGGGACTGAGCCTGA-3′ (SEQ ID NO: 54), 5′-AGGGGACTGAGCCTGA-3′ (SEQ ID NO: 55), 5′-GAGGGGACTGAGCCTGA-3′ (SEQ ID NO: 56), 5′-GGAGGGGACTGAGCCTGA-3′ (SEQ ID NO: 57), 5′-GGGAGGGGACTGAGCCTGA-3′ (SEQ ID NO: 58), 5′-CGGGAGGGGACTGAGCCTGA-3′ (SEQ ID NO: 59), 5′-TCGGGAGGGGACTGAGCCTGA-3′ (SEQ ID NO: 60), and 5′-ATCGGGAGGGGACTGAGCCTGA-3′ (SEQ ID NO: 61).

12. The oligonucleotide of any one of claims 1 to 11, wherein the oligonucleotide has at least about 50%, at least about 55%, at least about 60%, at least about 65%, at least about 70%, at least about 75%, at least about 80%, at least about 85%, at least about 90%, or at least about 95% sequence identity to 5′-AUCGGGAGGGGACUGAGCCUGA-3′ (SEQ ID NO: 31) or 5′-ATCGGGAGGGGACTGAGCCTGA-3′ (SEQ ID NO: 61).

13. The oligonucleotide of any one of claims 1 to 11, wherein the oligonucleotide has at least about 90% sequence similarity to 5′-AUCGGGAGGGGACUGAGCCUGA-3′ (SEQ ID NO: 31) or 5′-ATCGGGAGGGGACTGAGCCTGA-3′ (SEQ ID NO: 61).

14. The oligonucleotide of any one of claims 1 to 11, wherein the oligonucleotide comprises the nucleotide sequence as set forth in 5′-AUCGGGAGGGGACUGAGCCUGA-3′ (SEQ ID NO: 31) or 5′-ATCGGGAGGGGACTGAGCCTGA-3′ (SEQ ID NO: 61) with one substitution or two substitutions.

15. The oligonucleotide of any one of claims 1 to 11, wherein the oligonucleotide has the nucleotide sequence as set forth in 5′-AUCGGGAGGGGACUGAGCCUGA-3′ (SEQ ID NO: 31) or 5′-ATCGGGAGGGGACTGAGCCTGA-3′ (SEQ ID NO: 61).

16. The oligonucleotide of claim 15, wherein the oligonucleotide comprises the nucleotide sequence as set forth in 5′-AUCGGGAGGGGACUGAGCCUGA-3′ (SEQ ID NO: 62), wherein one or more of the bolded letters is a nucleic acid analog.

17. The oligonucleotide of claim 15 or 16, wherein the nucleotide sequence comprises 5′-AUCGGGAGGGGACUGAGCCUGA-3′ (SEQ ID NO: 63), and wherein one or more of the bolded letters are 2′-MOEs.

18. An oligonucleotide comprising the contiguous nucleotide sequence as set forth in 5′-AUCGGGAGGGGACUGAGCCUGA-3′ (SEQ ID NO: 64), wherein the bolded letters are 2′-MOEs.

19. The oligonucleotide of any one of claims 1 to 18, wherein the contiguous nucleotide sequence comprises one or more 5′ methyl pyrimidines.

20. The oligonucleotide of any one of claims 1 to 19, wherein the oligonucleotide comprises the contiguous nucleotide sequence as set forth in 5′-AUCGGGAGGGGACUGAGCCUGA-3′ (SEQ ID NO: 65), wherein one or more of the double underlined letters are 5-methyl pyrimidine.

21. The oligonucleotide of any one of claims 1 to 19, wherein the oligonucleotide comprises the contiguous nucleotide sequence as set forth in 5′-AUCGGGAGGGGACUGAGCCUGA-3′ (SEQ ID NO: 66), wherein the double underlined letters are 5-methyl pyrimidine.

22. An oligonucleotide comprising the contiguous nucleotide sequence as set forth in 5′-AUCGGGAGGGGACUGAGCCUGA-3′ (SEQ ID NO: 67), wherein the bolded letters are 2′-MOEs and the double underlined letters are 5-methyl pyrimidines.

23. The oligonucleotide of any one of claims 1 to 22, wherein the oligonucleotide further comprises a backbone modification.

24. The oligonucleotide of claim 23, wherein the backbone modification is a phosphorodiamidate morpholino oligomer (PMO) and/or phosphorothioate (PS) modification.

25. The oligonucleotide of claim 23 or 24, wherein each of the nucleotides are modified with PS modifications.

26. The oligonucleotide of claim 25, wherein the oligonucleotide comprises the contiguous nucleotide sequence 5′-AUCGGGAGGGGACUGAGCCUGA-3′ (SEQ ID NO: 73), wherein the bolded letters are 2′-MOEs and the double underlined letters are 5-methyl pyrimidines (ASO M4/miR-484-M4/miR-484 ASO M4).

27. The oligonucleotide of any one of claims 1 to 26, wherein the oligonucleotide is capable of reducing an mRNA level in a cell, at least about one fold, at least two folds, at least three folds, at least four folds, at least five folds, at least six folds, at least seven folds, at least eight folds, at least nine folds, or at least ten folds compared to the cell that is not treated with the oligonucleotide, wherein the mRNA encodes a proline-rich transmembrane protein 2 (PRRT2), an inward rectifier potassium channel 2 (KCNJ2), an activated CDC42 kinase 1 (TNK2), an ataxin-1-like (ATXN1), or any combination thereof.

28. The oligonucleotide of any one of claims 1 to 27, wherein the oligonucleotide is capable of reducing a protein expression in a cell at least about one fold, at least two folds, at least three folds, at least four folds, at least five folds, at least six folds, at least seven folds, at least eight folds, at least nine folds, or at least ten folds compared to the cell that is not treated with the oligonucleotide, wherein the protein comprises a V-type proton ATPase 116 kDa subunit a 2 (ATP6VOA2), a disintegrin and metalloproteinase domain-containing protein 10 (ADAM10), a protocadherin-19 (PCDH19), or any combination thereof.

29. The oligonucleotide of any one of claims 1 to 28, wherein the oligonucleotide is delivered via a delivery agent.

30. The oligonucleotide of claim 29, wherein the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

31. The oligonucleotide of any one of claims 1 to 30, which comprises an antisense oligonucleotide.

32. A delivery agent comprising the oligonucleotide of any one of claims 1 to 31.

33. The delivery agent of claim 32, which is a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

34. A pharmaceutical composition comprising (i) the oligonucleotide of any one of claims 1 to 31 or the delivery agent of claim 32 or 33 and (ii) a pharmaceutically acceptable carrier.

35. A method of preparing the oligonucleotide of any one of claims 1 to 31, the delivery agent of claim 32 or 33, and the pharmaceutical composition of claim 34.

36. A method of reducing an mRNA level in a cell comprising contacting the cell with the oligonucleotide of any one of claims 1 to 31, wherein the mRNA encodes a proline-rich transmembrane protein 2 (PRRT2), an inward rectifier potassium channel 2 (KCNJ2), an activated CDC42 kinase 1 (TNK2), an ataxin-1-like (ATXN1), or any combination thereof.

37. The method of claim 36, wherein the mRNA level is reduced at least about one fold, at least two folds, at least three folds, at least four folds, at least five folds, at least six folds, at least seven folds, at least eight folds, at least nine folds, or at least ten folds compared to the cell that is not contacted with the oligonucleotide.

38. A method of reducing a protein expression in a cell comprising contacting the cell with the oligonucleotide of any one of claims 1 to 31, wherein the protein comprises a V-type proton ATPase 116 kDa subunit a 2 (ATP6V0A2), a disintegrin and metalloproteinase domain-containing protein 10 (ADAM10), a protocadherin-19 (PCDH19), or any combination thereof.

39. The method of claim 38, wherein the protein expression is reduced at least about one fold, at least two folds, at least three folds, at least four folds, at least five folds, at least six folds, at least seven folds, at least eight folds, at least nine folds, or at least ten folds compared to the cell that is not contacted with the oligonucleotide.

40. A method of reducing an occurrence, a duration, or a symptom of a seizure in a subject in need thereof comprising administering an oligonucleotide comprising a contiguous nucleotide sequence of 10 to 30 nucleic acids in length that are complementary to a nucleic acid sequence within miRNA 484 (5′-UCAGGCUCAGUCCCCUCCCGAU-3′ (SEQ ID NO: 1)).

41. A method of treating an epilepsy in a subject in need thereof comprising administering an oligonucleotide comprising a contiguous nucleotide sequence of 10 to 30 nucleic acids in length that are complementary to a nucleic acid sequence within miRNA 484 (5′-UCAGGCUCAGUCCCCUCCCGAU-3′ (SEQ ID NO: 1)).

42. The method of claim 41, wherein the epilepsy comprises 1) focal onset seizure: atonic seizures, automatisms, clonic seizures, epileptic spasms, hyperkinetic seizures, myoclonic seizures, tonic seizures, autonomic seizures, behavior arrest, cognitive seizures, emotional seizures, sensory seizures. Focal onset seizure may be divided into focal onset aware seizure (simple partial seizure) and focal onset impaired awareness seizure (complex partial seizure) according to absence and presence of consciousness during seizure activities; 2) generalized onset seizure: myoclonic seizures, myoclonic-tonic-clonic seizures, tonic-clonic seizures, tonic seizure, clonic seizure, myoclonic-atonic seizure, typical absence seizures, atypical absence seizures, eyelid myoclonia; 3) unknown onset seizure (tonic-clonic seizure, epileptic spasms, behavior arrest), or 4) unclassified seizures.

43. The method of claim 41 or 42, wherein the oligonucleotide comprises an oligonucleotide of any one of claims 1 to 31.

44. The method of any one of claims 36 to 43, wherein the oligonucleotide is delivered via a delivery agent.

45. The method of claim 44, wherein the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

46. The method of any one of claims 36 to 45, comprising administering the delivery agent of any one of claims 32 or 33 or the pharmaceutical composition of claim 34.

47. The method of any one of claims 36 to 47, wherein the oligonucleotide is administered intranasally, parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracisternally, intracapsularly, intratumorally, topically, or any combination thereof.

48. The method of any one of claims 36 to 47, further comprising administering an anti-epileptic agent.


SEQUENCE LISTING (161 sequences)

SEQ ID NO: 1 - RNA, 22 nt, synthetic construct
  source 1..22: mol_type = other RNA; note = nucleic acid sequence within miRNA-484
tcaggctcagtcccctcccgat

SEQ ID NO: 2-5 - skipped (4 entries)

SEQ ID NO: 6 - RNA, 10 nt, synthetic construct
  source 1..10: mol_type = other RNA; note = Antisense Oligonucleotide
actgagcctg

SEQ ID NO: 7 - RNA, 11 nt, synthetic construct
  source 1..11: mol_type = other RNA; note = Antisense Oligonucleotide
gactgagcctg

SEQ ID NO: 8 - RNA, 12 nt, synthetic construct
  source 1..12: mol_type = other RNA; note = Antisense Oligonucleotide
ggactgagcctg

SEQ ID NO: 9 - RNA, 13 nt, synthetic construct
  source 1..13: mol_type = other RNA; note = Antisense Oligonucleotide
gggactgagcctg

SEQ ID NO: 10 - RNA, 14 nt, synthetic construct
  source 1..14: mol_type = other RNA; note = Antisense Oligonucleotide
ggggactgagcctg

SEQ ID NO: 11 - RNA, 15 nt, synthetic construct
  source 1..15: mol_type = other RNA; note = Antisense Oligonucleotide
aggggactgagcctg

SEQ ID NO: 12 - RNA, 16 nt, synthetic construct
  source 1..16: mol_type = other RNA; note = Antisense Oligonucleotide
gaggggactgagcctg

SEQ ID NO: 13 - RNA, 17 nt, synthetic construct
  source 1..17: mol_type = other RNA; note = Antisense Oligonucleotide
ggaggggactgagcctg

SEQ ID NO: 14 - RNA, 18 nt, synthetic construct
  source 1..18: mol_type = other RNA; note = Antisense Oligonucleotide
gggaggggactgagcctg

SEQ ID NO: 15 - RNA, 19 nt, synthetic construct
  source 1..19: mol_type = other RNA; note = Antisense Oligonucleotide
cgggaggggactgagcctg

SEQ ID NO: 16 - RNA, 20 nt, synthetic construct
  source 1..20: mol_type = other RNA; note = Antisense Oligonucleotide
tcgggaggggactgagcctg

SEQ ID NO: 17 - RNA, 21 nt, synthetic construct
  source 1..21: mol_type = other RNA; note = Antisense Oligonucleotide
atcgggaggggactgagcctg

SEQ ID NO: 18 - skipped

SEQ ID NO: 19 - RNA, 10 nt, synthetic construct
  source 1..10: mol_type = other RNA; note = Antisense Oligonucleotide
ctgagcctga

SEQ ID NO: 20 - RNA, 11 nt, synthetic construct
  source 1..11: mol_type = other RNA; note = Antisense Oligonucleotide
actgagcctga

SEQ ID NO: 21 - RNA, 12 nt, synthetic construct
  source 1..12: mol_type = other RNA; note = Antisense Oligonucleotide
gactgagcctga

SEQ ID NO: 22 - RNA, 13 nt, synthetic construct
  source 1..13: mol_type = other RNA; note = Antisense Oligonucleotide
ggactgagcctga

SEQ ID NO: 23 - RNA, 14 nt, synthetic construct
  source 1..14: mol_type = other RNA; note = Antisense Oligonucleotide
gggactgagcctga

SEQ ID NO: 24 - RNA, 15 nt, synthetic construct
  source 1..15: mol_type = other RNA; note = Antisense Oligonucleotide
ggggactgagcctga

SEQ ID NO: 25 - RNA, 16 nt, synthetic construct
  source 1..16: mol_type = other RNA; note = Antisense Oligonucleotide
aggggactgagcctga

SEQ ID NO: 26 - RNA, 17 nt, synthetic construct
  source 1..17: mol_type = other RNA; note = Antisense Oligonucleotide
gaggggactgagcctga

SEQ ID NO: 27 - RNA, 18 nt, synthetic construct
  source 1..18: mol_type = other RNA; note = Antisense Oligonucleotide
ggaggggactgagcctga

SEQ ID NO: 28 - RNA, 19 nt, synthetic construct
  source 1..19: mol_type = other RNA; note = Antisense Oligonucleotide
gggaggggactgagcctga

SEQ ID NO: 29 - RNA, 20 nt, synthetic construct
  source 1..20: mol_type = other RNA; note = Antisense Oligonucleotide
cgggaggggactgagcctga

SEQ ID NO: 30 - RNA, 21 nt, synthetic construct
  source 1..21: mol_type = other RNA; note = Antisense Oligonucleotide
tcgggaggggactgagcctga

SEQ ID NO: 31 - RNA, 22 nt, synthetic construct
  source 1..22: mol_type = other RNA; note = Antisense Oligonucleotide
atcgggaggggactgagcctga

SEQ ID NO: 32-35 - skipped (4 entries)

SEQ ID NO: 36 - DNA, 10 bp, synthetic construct
  source 1..10: mol_type = other DNA; note = Antisense Oligonucleotide
actgagcctg

SEQ ID NO: 37 - DNA, 11 bp, synthetic construct
  source 1..11: mol_type = other DNA; note = Antisense Oligonucleotide
gactgagcctg

SEQ ID NO: 38 - DNA, 12 bp, synthetic construct
  source 1..12: mol_type = other DNA; note = Antisense Oligonucleotide
ggactgagcctg

SEQ ID NO: 39 - DNA, 13 bp, synthetic construct
  source 1..13: mol_type = other DNA; note = Antisense Oligonucleotide
gggactgagcctg

SEQ ID NO: 40 - DNA, 14 bp, synthetic construct
  source 1..14: mol_type = other DNA; note = Antisense Oligonucleotide
ggggactgagcctg

SEQ ID NO: 41 - DNA, 15 bp, synthetic construct
  source 1..15: mol_type = other DNA; note = Antisense Oligonucleotide
aggggactgagcctg

SEQ ID NO: 42 - DNA, 16 bp, synthetic construct
  source 1..16: mol_type = other DNA; note = Antisense Oligonucleotide
gaggggactgagcctg

SEQ ID NO: 43 - DNA, 17 bp, synthetic construct
  source 1..17: mol_type = other DNA; note = Antisense Oligonucleotide
ggaggggactgagcctg

SEQ ID NO: 44 - DNA, 18 bp, synthetic construct
  source 1..18: mol_type = other DNA; note = Antisense Oligonucleotide
gggaggggactgagcctg

SEQ ID NO: 45 - DNA, 19 bp, synthetic construct
  source 1..19: mol_type = other DNA; note = Antisense Oligonucleotide
cgggaggggactgagcctg

SEQ ID NO: 46 - DNA, 20 bp, synthetic construct
  source 1..20: mol_type = other DNA; note = Antisense Oligonucleotide
tcgggaggggactgagcctg

SEQ ID NO: 47 - DNA, 21 bp, synthetic construct
  source 1..21: mol_type = other DNA; note = Antisense Oligonucleotide
atcgggaggggactgagcctg

SEQ ID NO: 48 - skipped

SEQ ID NO: 49 - DNA, 10 bp, synthetic construct
  source 1..10: mol_type = other DNA; note = Antisense Oligonucleotide
ctgagcctga

SEQ ID NO: 50 - DNA, 11 bp, synthetic construct
  source 1..11: mol_type = other DNA; note = Antisense Oligonucleotide
actgagcctga

SEQ ID NO: 51 - DNA, 12 bp, synthetic construct
  source 1..12: mol_type = other DNA; note = Antisense Oligonucleotide
gactgagcctga

SEQ ID NO: 52 - DNA, 13 bp, synthetic construct
  source 1..13: mol_type = other DNA; note = Antisense Oligonucleotide
ggactgagcctga

SEQ ID NO: 53 - DNA, 14 bp, synthetic construct
  source 1..14: mol_type = other DNA; note = Antisense Oligonucleotide
gggactgagcctga

SEQ ID NO: 54 - DNA, 15 bp, synthetic construct
  source 1..15: mol_type = other DNA; note = Antisense Oligonucleotide
ggggactgagcctga

SEQ ID NO: 55 - DNA, 16 bp, synthetic construct
  source 1..16: mol_type = other DNA; note = Antisense Oligonucleotide
aggggactgagcctga

SEQ ID NO: 56 - DNA, 17 bp, synthetic construct
  source 1..17: mol_type = other DNA; note = Antisense Oligonucleotide
gaggggactgagcctga

SEQ ID NO: 57 - DNA, 18 bp, synthetic construct
  source 1..18: mol_type = other DNA; note = Antisense Oligonucleotide
ggaggggactgagcctga

SEQ ID NO: 58 - DNA, 19 bp, synthetic construct
  source 1..19: mol_type = other DNA; note = Antisense Oligonucleotide
gggaggggactgagcctga

SEQ ID NO: 59 - DNA, 20 bp, synthetic construct
  source 1..20: mol_type = other DNA; note = Antisense Oligonucleotide
cgggaggggactgagcctga

SEQ ID NO: 60 - DNA, 21 bp, synthetic construct
  source 1..21: mol_type = other DNA; note = Antisense Oligonucleotide
tcgggaggggactgagcctga

SEQ ID NO: 61 - DNA, 22 bp, synthetic construct
  source 1..22: mol_type = other DNA; note = Antisense Oligonucleotide
atcgggaggggactgagcctga

SEQ ID NO: 62 - RNA, 22 nt, synthetic construct
  source 1..22: mol_type = other RNA; note = Antisense Oligonucleotide
  misc_difference order(1..3,13..14,18..22): note = one or more is a nucleic acid analog
atcgggaggggactgagcctga

SEQ ID NO: 63 - RNA, 22 nt, synthetic construct
  source 1..22: mol_type = other RNA; note = Antisense Oligonucleotide
  misc_difference order(1..3,13..14,18..22): note = one or more are 2'-O-methoxyethyl nucleic acids
atcgggaggggactgagcctga

SEQ ID NO: 64 - RNA, 22 nt, synthetic construct
  source 1..22: mol_type = other RNA; note = Antisense Oligonucleotide
  modified_base order(1..3,13..14,18..22): mod_base = OTHER; note = 2'-O-methoxyethyl nucleic acids
atcgggaggggactgagcctga

SEQ ID NO: 65 - RNA, 22 nt, synthetic construct
  source 1..22: mol_type = other RNA; note = Antisense Oligonucleotide
  misc_difference order(2..3,13..14,18..20): note = one or more are 5-methyl pyrimidines
atcgggaggggactgagcctga

SEQ ID NO: 66 - RNA, 22 nt, synthetic construct
  source 1..22: mol_type = other RNA; note = Antisense Oligonucleotide
  modified_base order(2..3,13..14,18..20): mod_base = OTHER; note = 5-methyl pyrimidines
atcgggaggggactgagcctga

SEQ ID NO: 67 - RNA, 22 nt, synthetic construct
  source 1..22: mol_type = other RNA; note = Antisense Oligonucleotide
  modified_base order(2..3,13..14,18..20): mod_base = OTHER; note = 5-methyl pyrimidines
  modified_base order(1..3,13..14,18..22): mod_base = OTHER; note = 2'-O-methoxyethyl nucleic acids
atcgggaggggactgagcctga

SEQ ID NO: 68 - RNA, 22 nt, synthetic construct
  source 1..22: mol_type = other RNA; note = Antisense Oligonucleotide
  misc_difference order(1..3,18..22): note = one or more is a nucleic acid analog
  modified_base 1..22: mod_base = OTHER; note = phosphorothioate backbone
atcgggaggggactgagcctga

SEQ ID NO: 69 - RNA, 22 nt, synthetic construct
  source 1..22: mol_type = other RNA; note = Antisense Oligonucleotide
  modified_base 1..22: mod_base = OTHER; note = phosphorothioate backbone
  misc_difference order(1..3,13..14,18..22): note = one or more are 2'-O-methoxyethyl nucleic acids
atcgggaggggactgagcctga

SEQ ID NO: 70 - RNA, 22 nt, synthetic construct
  source 1..22: mol_type = other RNA; note = Antisense Oligonucleotide
  modified_base order(1..3,13..14,18..22): mod_base = OTHER; note = 2'-O-methoxyethyl nucleic acids
  modified_base 1..22: mod_base = OTHER; note = phosphorothioate backbone
atcgggaggggactgagcctga

SEQ ID NO: 71 - RNA, 22 nt, synthetic construct
  source 1..22: mol_type = other RNA; note = Antisense Oligonucleotide
  misc_difference order(2..3,13..14,18..20): note = one or more are 5-methyl pyrimidines
  modified_base 1..22: mod_base = OTHER; note = phosphorothioate backbone
atcgggaggggactgagcctga

SEQ ID NO: 72 - RNA, 22 nt, synthetic construct
  source 1..22: mol_type = other RNA; note = Antisense Oligonucleotide
  modified_base order(2..3,13..14,18..20): mod_base = OTHER; note = 5-methyl pyrimidines
  modified_base 1..22: mod_base = OTHER; note = phosphorothioate backbone
atcgggaggggactgagcctga

SEQ ID NO: 73 - RNA, 22 nt, synthetic construct
  source 1..22: mol_type = other RNA; note = Antisense Oligonucleotide
  modified_base order(2..3,13..14,18..20): mod_base = OTHER; note = 5-methyl pyrimidines
  modified_base order(1..3,13..14,18..22): mod_base = OTHER; note = 2'-O-methoxyethyl nucleic acids
  modified_base 1..22: mod_base = OTHER; note = phosphorothioate backbone
atcgggaggggactgagcctga

SEQ ID NO: 74 - DNA, 22 bp, synthetic construct
  source 1..22: mol_type = other DNA; note = Antisense Oligonucleotide
cccgaggtttgcttttcatttg

SEQ ID NO: 75 - DNA, 22 bp, synthetic construct
  source 1..22: mol_type = other DNA; note = Antisense Oligonucleotide
ccacagtggttggcattcctga

SEQ ID NO: 76 - DNA, 22 bp, synthetic construct
  source 1..22: mol_type = other DNA; note = Antisense Oligonucleotide
ccacagtggttggcattcctga

SEQ ID NO: 77 - DNA, 24 bp, synthetic construct
  source 1..24: mol_type = other DNA; note = Antisense Oligonucleotide
gccagatttatgaagtactgagtg

SEQ ID NO: 78 - DNA, 22 bp, synthetic construct
  source 1..22: mol_type = other DNA; note = Antisense Oligonucleotide
ggcgatgacctcagcagagctt

SEQ ID NO: 79 - DNA, 22 bp, synthetic construct
  source 1..22: mol_type = other DNA; note = Antisense Oligonucleotide
gctgagcggcctgtcctccaag

SEQ ID NO: 80 - DNA, 22 bp, synthetic construct
  source 1..22: mol_type = other DNA; note = Antisense Oligonucleotide
gcccgccagcgcacacagagcc

SEQ ID NO: 81 - DNA, 22 bp, synthetic construct
  source 1..22: mol_type = other DNA; note = Antisense Oligonucleotide
cgaccacccggctgaggagagg

SEQ ID NO: 82 - DNA, 23 bp, synthetic construct
  source 1..23: mol_type = other DNA; note = Antisense Oligonucleotide
gttgacatactgctccagatcac

SEQ ID NO: 83 - DNA, 22 bp, synthetic construct
  source 1..22: mol_type = other DNA; note = Antisense Oligonucleotide
cacgttcgtcagagacatcgcg

SEQ ID NO: 84 - DNA, 22 bp, synthetic construct
  source 1..22: mol_type = other DNA; note = Antisense Oligonucleotide
ctcacgctgacgatctccgagc

SEQ ID NO: 85 - DNA, 23 bp, synthetic construct
  source 1..23: mol_type = other DNA; note = Antisense Oligonucleotide
gccataaaagacccttccaagtc

SEQ ID NO: 86 - DNA, 25 bp, synthetic construct
  source 1..25: mol_type = other DNA; note = Antisense Oligonucleotide
gagtctgaagactgcacgaaacttc

SEQ ID NO: 87 - DNA, 22 bp, synthetic construct
  source 1..22: mol_type = other DNA; note = Antisense Oligonucleotide
catgggaccccgtcgctttctg

SEQ ID NO: 88 - DNA, 22 bp, synthetic construct
  source 1..22: mol_type = other DNA; note = Antisense Oligonucleotide
ctgtaccaagaagcggccatcg

SEQ ID NO: 89 - DNA, 22 bp, synthetic construct
  source 1..22: mol_type = other DNA; note = Antisense Oligonucleotide
gtgaacagacgctgcgggaggt

SEQ ID NO: 90 - DNA, 24 bp, synthetic construct
  source 1..24: mol_type = other DNA; note = Antisense Oligonucleotide
gataccctgttgtccgtcttgatg

SEQ ID NO: 91 - DNA, 22 bp, synthetic construct
  source 1..22: mol_type = other DNA; note = Antisense Oligonucleotide
gatccttaacgaggcctaccgc

SEQ ID NO: 92 - DNA, 23 bp, synthetic construct
  source 1..23: mol_type = other DNA; note = Antisense Oligonucleotide
gtaacccatcgctacaaataagc

SEQ ID NO: 93 - DNA, 22 bp, synthetic construct
  source 1..22: mol_type = other DNA; note = Antisense Oligonucleotide
ctgaggctgtgatgggacttgg

SEQ ID NO: 94 - DNA, 21 bp, synthetic construct
  source 1..21: mol_type = other DNA; note = Antisense Oligonucleotide
cagtgggaggccatgttttag

SEQ ID NO: 95 - DNA, 23 bp, synthetic construct
  source 1..23: mol_type = other DNA; note = Antisense Oligonucleotide
cagacttcgtgaatgggaatttg

SEQ ID NO: 96 - DNA, 23 bp, synthetic construct
  source 1..23: mol_type = other DNA; note = Antisense Oligonucleotide
cagaggagttccttatgtcagag

SEQ ID NO: 97 - DNA, 22 bp, synthetic construct
  source 1..22: mol_type = other DNA; note = Antisense Oligonucleotide
cggatggggaggatggaagctt

SEQ ID NO: 98 - DNA, 22 bp, synthetic construct
  source 1..22: mol_type = other DNA; note = Antisense Oligonucleotide
gattggcattccgctggtccaa

SEQ ID NO: 99 - DNA, 23 bp, synthetic construct
  source 1..23: mol_type = other DNA; note = Antisense Oligonucleotide
gcggagcaagtacaatgatgaca

SEQ ID NO: 100 - DNA, 22 bp, synthetic construct
  source 1..22: mol_type = other DNA; note = Antisense Oligonucleotide
caatgagccgctccagttcctt

SEQ ID NO: 101 - DNA, 21 bp, synthetic construct
  source 1..21: mol_type = other DNA; note = Antisense Oligonucleotide
gtggtgtcaaggagtgggaag

SEQ ID NO: 102 - DNA, 21 bp, synthetic construct
  source 1..21: mol_type = other DNA; note = Antisense Oligonucleotide
catcaatgagccggtgctcat

SEQ ID NO: 103 - DNA, 22 bp, synthetic construct
  source 1..22: mol_type = other DNA; note = Antisense Oligonucleotide
gtcactgctgtatggactcatc

SEQ ID NO: 104 - DNA, 22 bp, synthetic construct
  source 1..22: mol_type = other DNA; note = Antisense Oligonucleotide
gaggacaatgaggacgtccctg

SEQ ID NO: 105 - DNA, 21 bp, synthetic construct
  source 1..21: mol_type = other DNA; note = Antisense Oligonucleotide
gttgaggagagtcccaaggtt

SEQ ID NO: 106 - DNA, 22 bp, synthetic construct
  source 1..22: mol_type = other DNA; note = Antisense Oligonucleotide
gattcccctcctgggcttaagc

SEQ ID NO: 107 - DNA, 23 bp, synthetic construct
  source 1..23: mol_type = other DNA; note = Antisense Oligonucleotide
gtcccactttgagtacgtcaaga

SEQ ID NO: 108 - DNA, 21 bp, synthetic construct
  source 1..21: mol_type = other DNA; note = Antisense Oligonucleotide
ggtgggaactcagcctccagt

SEQ ID NO: 109 - DNA, 23 bp, synthetic construct
  source 1..23: mol_type = other DNA; note = Antisense Oligonucleotide
caagtcaaggtccttgttgtaca

SEQ ID NO: 110 - DNA, 22 bp, synthetic construct
  source 1..22: mol_type = other DNA; note = Antisense Oligonucleotide
cgtacactcctctaagccatat

SEQ ID NO: 111 - DNA, 21 bp, synthetic construct
  source 1..21: mol_type = other DNA; note = Antisense Oligonucleotide
cgagaggcagagactatgcga

SEQ ID NO: 112 - DNA, 22 bp, synthetic construct
  source 1..22: mol_type = other DNA; note = Antisense Oligonucleotide
gagttgtccaaattcgtccgtt

SEQ ID NO: 113 - DNA, 21 bp, synthetic construct
  source 1..21: mol_type = other DNA; note = Antisense Oligonucleotide
cgtcatgcaatacgccgactc

SEQ ID NO: 114 - DNA, 22 bp, synthetic construct
  source 1..22: mol_type = other DNA; note = Antisense Oligonucleotide
gtacgggtgaggaaccgacttg

SEQ ID NO: 115 - DNA, 23 bp, synthetic construct
  source 1..23: mol_type = other DNA; note = Antisense Oligonucleotide
ccaaggaaaagtggaagcatttg

SEQ ID NO: 116 - DNA, 22 bp, synthetic construct
  source 1..22: mol_type = other DNA; note = Antisense Oligonucleotide
ggctgtgtttggataggggtac

SEQ ID NO: 117 - DNA, 22 bp, synthetic construct
  source 1..22: mol_type = other DNA; note = Antisense Oligonucleotide
gtatcttacgacgactgcactg

SEQ ID NO: 118 - DNA, 21 bp, synthetic construct
  source 1..21: mol_type = other DNA; note = Antisense Oligonucleotide
cttccttggcaagcctgaatg

SEQ ID NO: 119 - DNA, 22 bp, synthetic construct
  source 1..22: mol_type = other DNA; note = Antisense Oligonucleotide
gccagtgactgtccttacagtg

SEQ ID NO: 120 - DNA, 22 bp, synthetic construct
  source 1..22: mol_type = other DNA; note = Antisense Oligonucleotide
gtttctacaccacggatgtcag

SEQ ID NO: 121 - DNA, 20 bp, synthetic construct
  source 1..20: mol_type = other DNA; note = Antisense Oligonucleotide
ctgaagctgtgaaccggtcc

SEQ ID NO: 122 - DNA, 21 bp, synthetic construct
  source 1..21: mol_type = other DNA; note = Antisense Oligonucleotide
gagagccatcagtccccaggc

SEQ ID NO: 123 - DNA, 20 bp, synthetic construct
  source 1..20: mol_type = other DNA; note = Antisense Oligonucleotide
gcagtcagtcatcggtcaca

SEQ ID NO: 124 - DNA, 20 bp, synthetic construct
  source 1..20: mol_type = other DNA; note = Antisense Oligonucleotide
gacagaggtggaagagggtg

SEQ ID NO: 125 - DNA, 20 bp, synthetic construct
  source 1..20: mol_type = other DNA; note = Antisense Oligonucleotide
gccatctgggttcgaggaag

SEQ ID NO: 126 - DNA, 22 bp, synthetic construct
  source 1..22: mol_type = other DNA; note = Antisense Oligonucleotide
cgagagggattgtcacagaatg

SEQ ID NO: 127 - DNA, 20 bp, synthetic construct
  source 1..20: mol_type = other DNA; note = Antisense Oligonucleotide
ctgcaggcagttcttccctc

SEQ ID NO: 128 - DNA, 20 bp, synthetic construct
  source 1..20: mol_type = other DNA; note = Antisense Oligonucleotide
gtccagtgcgccctcgattc

SEQ ID NO: 129 - DNA, 19 bp, synthetic construct
  source 1..19: mol_type = other DNA; note = Antisense Oligonucleotide
ctagagcagcccgggtcct

SEQ ID NO: 130 - DNA, 20 bp, synthetic construct
  source 1..20: mol_type = other DNA; note = Antisense Oligonucleotide
gcagcagtgtccactgaatt

SEQ ID NO: 131 - DNA, 21 bp, synthetic construct
  source 1..21: mol_type = other DNA; note = Antisense Oligonucleotide
ctggtatcccctgcatacacc

SEQ ID NO: 132 - DNA, 21 bp, synthetic construct
  source 1..21: mol_type = other DNA; note = Antisense Oligonucleotide
catccaacactcctgaccagg

SEQ ID NO: 133 - DNA, 22 bp, synthetic construct
  source 1..22: mol_type = other DNA; note = Antisense Oligonucleotide
gtattggaacgggaatttcagc

SEQ ID NO: 134 - DNA, 22 bp, synthetic construct
  source 1..22: mol_type = other DNA; note = Antisense Oligonucleotide
caggactctgctcataagttcg

SEQ ID NO: 135 - DNA, 22 bp, synthetic construct
  source 1..22: mol_type = other DNA; note = Antisense Oligonucleotide
gagagcaattaagatgggtgca

SEQ ID NO: 136 - DNA, 23 bp, synthetic construct
  source 1..23: mol_type = other DNA; note = Antisense Oligonucleotide
cttgagttagttaggctggggtt

SEQ ID NO: 137 - DNA, 21 bp, synthetic construct
  source 1..21: mol_type = other DNA; note = Antisense Oligonucleotide
ctacagccttccgtgagaagc

SEQ ID NO: 138 - DNA, 22 bp, synthetic construct
  source 1..22: mol_type = other DNA; note = Antisense Oligonucleotide
cacacttgatcttgccagcatg

SEQ ID NO: 139 - DNA, 22 bp, synthetic construct
  source 1..22: mol_type = other DNA; note = Antisense Oligonucleotide
gaatgaacatgggatgaccctg

SEQ ID NO: 140 - DNA, 24 bp, synthetic construct
  source 1..24: mol_type = other DNA; note = Antisense Oligonucleotide
cacttttccattggtctgacacta

SEQ ID NO: 141 - DNA, 21 bp, synthetic construct
  source 1..21: mol_type = other DNA; note = Antisense Oligonucleotide
gacctccttcctctacctacc

SEQ ID NO: 142 - DNA, 20 bp, synthetic construct
  source 1..20: mol_type = other DNA; note = Antisense Oligonucleotide
gttcctggtcagtcccgctg

SEQ ID NO: 143 - DNA, 23 bp, synthetic construct
  source 1..23: mol_type = other DNA; note = Antisense Oligonucleotide
gaccttagtgtgaggctttcagg

SEQ ID NO: 144 - DNA, 19 bp, synthetic construct
  source 1..19: mol_type = other DNA; note = Antisense Oligonucleotide
caggcgttccagctccttg

SEQ ID NO: 145 - DNA, 22 bp, synthetic construct
  source 1..22: mol_type = other DNA; note = Antisense Oligonucleotide
cattaccgagaacaccagaagg

SEQ ID NO: 146 - DNA, 20 bp, synthetic construct
  source 1..20: mol_type = other DNA; note = Antisense Oligonucleotide
ggagctgagtagtgtccacc

SEQ ID NO: 147 - DNA, 22 bp, synthetic construct
  source 1..22: mol_type = other DNA; note = Antisense Oligonucleotide
ctctattggcatccgtcatgtg

SEQ ID NO: 148 - DNA, 20 bp, synthetic construct
  source 1..20: mol_type = other DNA; note = Antisense Oligonucleotide
ccggtcatgcttaggtagca

SEQ ID NO: 149 - DNA, 21 bp, synthetic construct
  source 1..21: mol_type = other DNA; note = Antisense Oligonucleotide
ccaggcattctgaagaaggac

SEQ ID NO: 150 - DNA, 21 bp, synthetic construct
  source 1..21: mol_type = other DNA; note = Antisense Oligonucleotide
ctcagacccttcctctgctgc

SEQ ID NO: 151 - DNA, 22 bp, synthetic construct
  source 1..22: mol_type = other DNA; note = Antisense Oligonucleotide
gccctcaagacatctacaatgt

SEQ ID NO: 152 - DNA, 21 bp, synthetic construct
  source 1..21: mol_type = other DNA; note = Antisense Oligonucleotide
cgacatttcgagggaagggtc

SEQ ID NO: 153 - DNA, 19 bp, synthetic construct
  source 1..19: mol_type = other DNA; note = Antisense Oligonucleotide
gtgagcaggagaggctgtg

SEQ ID NO: 154 - DNA, 23 bp, synthetic construct
  source 1..23: mol_type = other DNA; note = Antisense Oligonucleotide
caactacctgctgagcaaatcac

SEQ ID NO: 155 - DNA, 20 bp, synthetic construct
  source 1..20: mol_type = other DNA; note = Antisense Oligonucleotide
caatgcggcttctgtgagcc

SEQ ID NO: 156 - DNA, 20 bp, synthetic construct
  source 1..20: mol_type = other DNA; note = Antisense Oligonucleotide
catcagctagctcccacgga

SEQ ID NO: 157 - DNA, 24 bp, synthetic construct
  source 1..24: mol_type = other DNA; note = Antisense Oligonucleotide
gtggttgtgcaatatagtgatgcc

SEQ ID NO: 158 - DNA, 22 bp, synthetic construct
  source 1..22: mol_type = other DNA; note = Antisense Oligonucleotide
gagggtgtgctgctattaggca

SEQ ID NO: 159 - DNA, 20 bp, synthetic construct
  source 1..20: mol_type = other DNA; note = Antisense Oligonucleotide
gttggtggctctcacggtgc

SEQ ID NO: 160 - DNA, 20 bp, synthetic construct
  source 1..20: mol_type = other DNA; note = Antisense Oligonucleotide
gcctgcgtcatcaggatctc

SEQ ID NO: 161 - DNA, 22 bp, synthetic construct
  source 1..22: mol_type = other DNA; note = Antisense Oligonucleotide
agaagagggtcatcctgggaga